SEQUENCE LISTING (130 sequences)

SEQ ID NO: 1 - protein, 1228 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Candidatus Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, Lachnospiraceae bacterium
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, Lachnospiraceae bacterium
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Lachnospiraceae bacterium
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, Peregrinibacteria bacterium
  misc_feature (1073)..(1073): Xaa can be any naturally occurring amino acid
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, Acidaminococcus sp.
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, Acidaminococcus sp.
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, Acidaminococcus sp.
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, Artificial sequence
  Synthetic polypeptide
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, Artificial sequence
  Synthetic polypeptide
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, Artificial sequence
  Synthetic polypeptide
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, Artificial sequence
  Synthetic polypeptide
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, Artificial sequence
  Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, Artificial sequence
  Synthetic polypeptide
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, Artificial sequence
  Synthetic polypeptidfe
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, Artificial sequence
  Synthetic polypeptide
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 83 aa, Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 36 - DNA, 19 bp, Artificial sequence
  Synthetic oligonucleotide
  misc_feature (1)..(19): n is a, c, g, or t
nnnnnnnnnnnnnnnnnnn

SEQ ID NO: 37 - DNA, 22 bp, Artificial sequence
  Synthetic oligonucleotide
  misc_feature (4)..(22): n is a, c, g, or t
aaannnnnnnnnnnnnnnnnnn

SEQ ID NO: 38 - DNA, 22 bp, Artificial sequence
  Synthetic oligonucleotide
  misc_feature (4)..(22): n is a, c, g, or t
tttnnnnnnnnnnnnnnnnnnn

SEQ ID NO: 39 - protein, 24 aa, Artificial sequence
  Synthetic peptide
EELLSKNYHLENEVARLKKGSGSG

SEQ ID NO: 40 - protein, 241 aa, Artificial sequence
  Synthetic polypeptide
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 41 - protein, 277 aa, Artificial sequence
  Synthetic polypeptide
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 42 - DNA, 66 bp, Artificial sequence
  Synthetic oligonucleotide
ttcttgtcgtacttatagatcgctacgttatttcaattttgaaaatctgagtcctgggagtgcgga

SEQ ID NO: 43 - protein, 605 aa, Artificial sequence
  Synthetic polypeptide
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 44 - protein, 482 aa, Artificial sequence
  Synthetic polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 45 - DNA, 10 bp, Artificial sequence
  Synthetic oligonucleotide
aatttttgga

SEQ ID NO: 46 - protein, 83 aa, Artificial sequence
  Synthetic polypeptide
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 47 - DNA, 25 bp, Artificial sequence
  Synthetic oligonucleotide
gcgcacatgaggatcacccatgtgc

SEQ ID NO: 48 - protein, 116 aa, Artificial sequence
  Synthetic polypeptide
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 49 - DNA, 26 bp, Artificial sequence
  Synthetic oligonucleotide
ataaggagtttatatggaaaccctta

SEQ ID NO: 50 - protein, 127 aa, Artificial sequence
  Synthetic polypeptide
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 51 - DNA, 19 bp, Artificial sequence
  Synthetic oligonucleotide
ctgaatgcctgcgagcatc

SEQ ID NO: 52 - protein, 62 aa, Artificial sequence
  Synthetic polypeptide
MKSIRCKNCNKLLFKADSFDHIEIRCPRCKRHIIMLNACEHPTEKHCGKREKITHSDETVRY

SEQ ID NO: 53 - DNA, 23 bp, Artificial sequence
  Synthetic oligonucleotide
cccaaaaggacagacaacgagat

SEQ ID NO: 54 - DNA, 23 bp, Artificial sequence
  Synthetic oligonucleotide
ggcaaatgatttgctatcgctga

SEQ ID NO: 55 - DNA, 23 bp, Artificial sequence
  Synthetic oligonucleotide
gtagttcttgatctcgttgtctg

SEQ ID NO: 56 - DNA, 23 bp, Artificial sequence
  Synthetic oligonucleotide
agtagttcttgatctcgttgtct

SEQ ID NO: 57 - protein, 413 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTTNNHPLQLQHHHQLVNINPSTQTHGVVMTKAAATTSSLPCLDVLKAWQGAWSSKPIIRDNCSSNTIVSGMNMMMAMDDLESPTSTLNFPAAYNATYPMQSLHGKQLPQFKEELDNTMVLQEIHAYNGCTDESTTTNAWFAEDGLIRSNGGNDGSSNVILPAAAAALSNIMEGFCGTADMLVYNSDEQNIAAGEISDNGGGGGGDGSCGRSGSNVEESRSYWDSILNLVNASPSGSPVF

SEQ ID NO: 58 - protein, 401 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTTNNHPLQLQHHHQLVNINPSTQTHGVVMTKAAATTPSLPCLDVLKAWQGAWSSKPIIRDNCSSNTIVSGMNMIMAMDDLESPTSTLNFPTAYNATYPMHYLNGKQFPQFKEELDNTMVLQEIHAYNGCTDESTTTNGGNDGSRNVILPAAAAALSNIMEGFCGTADMLVYNSDEQNIAAGEISYNDGGGGGDGSCGRSGSNVEESRSYWDSILNLVNASPSGSPVF

SEQ ID NO: 59 - protein, 413 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTNNNHPLQLQLQHHHQLVNINPSTQTHGVVMTKAAAATSSLPCLDVLKAWQGAWSSKPIIRDNCSSNTIVSGMNMMMAMDDLESPTSTLNFPAAYNATYPMQSLNGKQLPQFKEELDNTMVLQEIHAYNGCTDESTTTHAWFAEDGLIRSNGGNDGSSNVILPAAAAALSNIMEGFCGTADMLVYNSDEQNIAAGEISDNGGGGDGSCGRSGSNVEENRSYWDSILNLVNASPSGSPVF

SEQ ID NO: 60 - protein, 413 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTNNNHPLQLQLQHHHQLVNINPSTQTHGVVMTKAAAATSSLPCLDVLKAWQGAWSAKPIIRDNCSSNTIVSGMNMMMAMDDLESPTSTLNFPAAYNATYPMQSLNGKQLPQFKEELDNTMVLQEIHAYNGCTDESTTTHAWFAEDGLIRSNGGNDGSSNVILPAAAAALSNIMEGFCGTADMLVYNSDEQNIAAGEISDNSGGGDGSCGRSGSNVEENRSYWDSILNLVNASPSGSPVF

SEQ ID NO: 61 - protein, 417 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTTNNHPLQLQLQHHHQLVNINPSTQTHGVVMTKAAATTSSLPCLDVLKAWQGAWSSKPIIRDNCSSNTIVSGMNMMMAMDDLESPTSTLNFPAVYNATYPMQSLNGKQLPQFKEELDSTMVLQEIHAYNGCTDESTTTNAWFAEDSLIRSNGGNDGSSNVILPAAAAALSNIMEGFCGTADMLVYNSDEQNIAAGEISDNGGGGGGGGDGSCGRSGSNVEESRSYWDSILNLVNASPSGSPVF

SEQ ID NO: 62 - protein, 401 aa, Rubus sp.
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTTNNHPLQLQHHHQLVNINPSTQTHGVVMTKAAATTPSLPCLDVLKAWQGAWSSKPIIRDNCSSNTIVSGMNMIMAMDDLESPTSTLNFPTAYNATYPMHYLNGKQFPQFKEELDNTMVLQEIHAYNGCTDESTTTNGGNDGPRNVILPAAAAALSNIMEGFCGTADMLVYNSDEQNIAAGEISYNDGGGSGDGSCGRSGSNVEESRSYWDSILNLVNASPSGSPVF

SEQ ID NO: 63 - DNA, 1242 bp, Rubus sp.
atggggaggtctccatgttgtgataaggtgggtttgaaaaaaggaccatggactcctgaggaagaccaaaagcttttgtcttacatccaagagtatggccatggaagctggcgtgctttgccggcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccatgatgcatacggttccggcagcggccacgctaaggacgctgccaacctgagccacatggcccagtgggagagcgcccgtctcgaagccgaggccaggctggtcagagaatctaagcagctgatcatgaccaccaccaacaatcacccgctccaactccaacaccaccaccagctcgtcaacatcaacccctccacccaaactcacggagtagtcatgaccaaagccgccgccaccacgtcatcgctgccgtgtctggatgtgctgaaagcttggcaaggggcgtggtcgtcaaagcccataattcgtgacaactgtagtagtaacacaattgtttcgggtatgaatatgatgatggccatggatgacctcgagtctccaacatccacgttgaacttcccggcagcgtacaacgcaacatatccaatgcaatcccttcacggaaagcaattgccccagttcaaagaagagctggacaacaccatggtgctgcaggagattcatgcatacaacggttgcacggatgaaagtacaactactaatgcgtggttcgcggaggacggtttgattaggtcaaatggtggaaacgatggctcgagcaatgtgattctgccggctgctgctgctgcactttccaatatcatggagggtttctgtggcaccgcggatatgttggtttacaactcggatgagcagaacatagcggccggggagatctcagataatggcggaggcggaggcggagatggaagctgtgggcgtagtgggagcaacgttgaggagagcaggagctactgggacagtatactgaatttggtgaatgcttctccatccggctcgcctgtgttttaa

SEQ ID NO: 64 - DNA, 1206 bp, Rubus sp.
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccatgatgcatacggttccggtagcggccacgctaaggacgctgccaacctgagccacatggcccagtgggagagcgcccgtctcgaagccgaggccaggctggtcagagaatctaagcagctgatcatgaccaccaccaacaatcaccctctccaactccaacaccaccaccagctcgtcaacatcaacccctccacccaaactcacggagtagtcatgaccaaagccgccgccaccacgccatcgctgccgtgtctggatgtgctgaaagcttggcaaggggcgtggtcgtcaaagcccataattcgtgacaactgtagtagtaacacaattgtttcgggtatgaatatgattatggccatggacgacctcgagtctccaacatccacgttgaacttccccacagcgtacaacgcaacatatccaatgcattaccttaacggaaagcaatttccccagttcaaagaagagctggacaacaccatggtgctgcaggagattcatgcatacaacggttgcacggatgaaagtacaactacaaatggtggaaacgatggctcgagaaatgtgattctgccggctgctgctgctgcactttccaatatcatggagggtttctgtggcaccgcggatatgttggtttacaactcggatgagcagaacatagcggccggggagatctcatataatgacggaggcggaggcggagatggaagctgtgggcgtagtgggagcaacgttgaggagagcaggagctactgggacagtatactgaatttggtgaatgcttctccatccggctcgcctgtgttttaa

SEQ ID NO: 65 - DNA, 1242 bp, Rubus sp.
atggggaggtctccatgttgtgataaagtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccacgatgcatacggttccggcagcggccacgccaaggacgctgccaacctcagccacatggcccagtgggagagcgccaggctggaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcttcaccatccggttcgcctgtgttttaa

SEQ ID NO: 66 - DNA, 1242 bp, Rubus sp.
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaataacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcggcaaagcccataattcgtgacaactgtagtagtaacacaattgtttcgggtatgaatatgatgatggccatggacgacctcgagtcgccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatataatggagggtttctgtggcaccgcggatatgttggtttacaactcggatgagcagaacatagcggccggggagatctcagataatagcggaggcggagatggaagctgtgggcgtagtggcagcaacgttgaggagaacaggagctactgggacagtatactgaatttggtgaatgcttcaccatccggctcgcctgtgttttga

SEQ ID NO: 67 - DNA, 1254 bp, Rubus sp.
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccatgatgcatacggttccggcagcggccacgctaaggacgctgccaacctgagccacatggcccagtgggagagcgcccgtctcgaagccgaggccaggctggtcagagaatctaagcagctgatcatgaccaccaccaacaatcaccctctccaactccaactccaacaccaccaccagctcgtcaacatcaacccctccacccaaactcacggagtagtcatgaccaaagccgccgccaccacgtcatcgctgccgtgtctggatgtgctgaaagcttggcaaggggcgtggtcgtcaaagcccataattcgtgacaactgtagtagtaacacaattgtttcgggtatgaatatgatgatggccatggacgacctcgagtctccaacatccacgttgaacttcccggcagtgtacaacgcaacatatccaatgcaatcccttaacggaaagcaattgccccagttcaaagaagagctggacagcaccatggtgctgcaggagattcatgcatacaacggttgcacggatgaaagtacaactactaatgcgtggttcgcggaggacagtttgattaggtcaaatggtggaaacgatggctcgagcaatgtgattctgccggctgctgctgctgcactttccaatatcatggagggtttctgtggcaccgcggatatgttggtttacaactcggatgagcagaacatagcggccggggagatctcagataatggcggaggaggaggtggaggcggagatggaagctgtgggcgtagtgggagcaacgttgaggagagcaggagctactgggacagtatactgaatttggtgaatgcttctccatccggctcgcctgtgttttaa

SEQ ID NO: 68 - DNA, 1206 bp, Rubus sp.
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccatgatgcatacggttccggtagcggccacgctaaggacgctgccaacctgagccacatggcccagtgggagagcgcccgtctcgaagccgaggccaggctggtcagagaatctaagcagctgatcatgaccaccaccaacaatcaccctctccaactccaacaccaccaccagctcgtcaacatcaacccctccacccaaactcacggagtagtcatgaccaaagccgccgccaccacgccatcgctgccttgtctggatgtgctgaaagcttggcaaggggcgtggtcgtcaaagcccataattcgtgacaactgtagtagtaacacaattgtttcgggtatgaatatgattatggccatggacgacctcgagtctccaacatccacgttgaacttccccacagcgtacaacgcaacatatccaatgcattaccttaacggaaagcaatttccccagttcaaagaagagctggacaacaccatggtgctgcaggagattcatgcatacaacggttgcacggatgaaagtacaactacaaatggtggaaacgatggcccgagaaatgtgattctgccggctgctgctgctgcactttccaatatcatggagggtttctgtggcaccgcggatatgttggtttacaactcggatgagcagaacatagcggccggggagatctcatataatgacggaggcggaagcggagatggaagctgtgggcgtagtgggagcaacgttgaggagagcaggagctactgggacagtatactgaatttggtgaatgcttctccatccggctcgcctgtgttttaa

SEQ ID NO: 69 - DNA, 1242 bp, Rubus occidentalis
atggggaggtctccatgttgtgataaggtgggtttgaaaaaaggaccatggactcctgaggaagaccaaaagcttttgtcttacatccaagagtatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 70 - DNA, 1242 bp, Rubus idaeus
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccacgatgcatacggttccggcagcggccacgccaaggacgctgccaacctcagccacatggcccagtgggagagcgccaggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcttcaccatccggctcgcctgtgttttaa

SEQ ID NO: 71 - protein, 413 aa, Rubus occidentalis; identical to SEQ ID NO: 59

SEQ ID NO: 72 - protein, 413 aa, Rubus idaeus; identical to SEQ ID NO: 59

SEQ ID NO: 73 - protein, 18 aa, Artificial sequence
  Synthetic peptide
PKRTDNEIKNYWNTHLKK

SEQ ID NO: 74 - protein, 36 aa, Artificial sequence
  Synthetic peptide
SAIANHLPKRTDNEIKNYWNTHLKKRLTKMGIDPVT

SEQ ID NO: 75 - protein, 45 aa, Artificial sequence
  Synthetic peptide
TIIQLHALLGNRWSAIANHLPKRTDNEIKNYWNTHLKKRLTKMGI

SEQ ID NO: 76 - protein, 49 aa, Artificial sequence
  Synthetic peptide
TIIQLHALLGNRWSAIANHLPKRTDNEIKNYWNTHLKKRLTKMGIDPVT

SEQ ID NO: 77 - DNA, 80 bp, Artificial sequence
  Synthetic oligonucleotide
cttctcggaaataggtggtcagcgatagcaaatcatttgcccaaaaggacagacaacgagatcaagaactactggaacac

SEQ ID NO: 78 - DNA, 390 bp, Artificial sequence
  Synthetic oligonucleotide
atggggaggtctccatgttgtgataaggtgggtttgaaaaaaggaccatggactcctgaggaagaccaaaagcttttgtcttacatccaagagtatggccatggaagctggcgtgctttgccggcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcac

SEQ ID NO: 79 - protein, 1367 aa, Artificial sequence
  Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 80 - protein, 1367 aa, Artificial sequence
  Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 81 - DNA, 80 bp, Artificial sequence; identical to SEQ ID NO: 77
  Synthetic oligonucleotide

SEQ ID NO: 82 - protein, 26 aa, Artificial sequence
  Synthetic peptide
LLGNRWSAIANHLPKRTDNEIKNYWN

SEQ ID NO: 83 - DNA, 390 bp, Artificial sequence
  Synthetic polynucleotide
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcac

SEQ ID NO: 84 - DNA, 81 bp, Artificial sequence
  Synthetic oligonucleotide
aggaccatggactcctgaggaagaccaaaagcttttgtcttacatccaagagtatggccatggaagctggcgtgctttgcc

SEQ ID NO: 85 - DNA, 81 bp, Artificial sequence
  Synthetic oligonucleotide
aggaccatggactcctgaggaagaccaaaagcttttgtcttacatccaagagtatgggcatggaagctggcgtgctttgcc

SEQ ID NO: 86 - DNA, 57 bp, Artificial sequence
  Synthetic oligonucleotide
aggaccatggactcctgaggaagaccaaaagcttttgtcttacatccaagagtatgg

SEQ ID NO: 87 - DNA, 53 bp, Artificial sequence
  Synthetic oligonucleotide
tgaggaagaccaaaagcttttgtcttacatccaagagtatggccatggaagct

SEQ ID NO: 88 - DNA, 29 bp, Artificial sequence
  Synthetic oligonucleotide
aaagcttttgtcttacatccaagagtatg

SEQ ID NO: 89 - DNA, 30 bp, Artificial sequence
  Synthetic oligonucleotide
aataggtggtcagcgatagcaaatcatttg

SEQ ID NO: 90 - protein, 28 aa, Artificial sequence
  Synthetic peptide
WTPEEDQKLLSYIQEYGHGSWRALPAKA

SEQ ID NO: 91 - protein, 13 aa, Artificial sequence
  Synthetic peptide
KLLSYIQEYGHGS

SEQ ID NO: 92 - protein, 10 aa, Artificial sequence
  Synthetic peptide
NRWSAIANHL

SEQ ID NO: 93 - DNA, 1248 bp, Artificial sequence
  Synthetic polynucleotide
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacwgacaacgagatcaagaactactggaacacgcacctcaagaaragactgaccaagatgggcatcgaccccgtgacccacaagcccaagcaccatgatgcatacggttccggcagcggccacgctaaggacgctgcyaacctgagccacatggcccagtgggagagcgcccgtctcgaagccgaggccaggctsgtcagagaatctaagcagctratcatgaccaccaccaacaatcacccgctccatctccaacaccaccaccagctcgtcaacatcaacccctccacccaaactcayggagtagtcatgaccaaagccgccgccaccacgtcatcgctgccgtgtctggatgtgctgaaagcttggcaaggggcgtggtcrtcaaagcccataattcgtgacaactgyagtartaacacaattgtttcgggtatgaatatgatgatggccatggacgacctcgagtctccaacatccackttgaacttcccggcagcgtacaacgcaacatatccaatgcawtcccttaacggaaagcaattgccccagttcaaagaagagctggacaacaccatggtgctgcargagattcatscatacaacggttgcacggatgaaagtacaactactaatgcgtggttcgcggaggacggtttgattaggtcaaatggtggaaacgatggctcgagcartgtgattctgccggctgctgcttctgcactttccaatatcatggagggtttctgtggcaccgcggatatgttggtttacaactcggatgagcagaacatagcggccggggagatctcagataatggcggaggaggaggtggaggcggagatggaagctgtgggcgtagtgggagcaacgttgaggaragcaggagctactgggacagtatactgaatttggtgaatgcttctccatccggctcgcctgtgttttaa

SEQ ID NO: 94 - protein, 415 aa, Artificial sequence
  Synthetic polypeptide
  misc_feature (238)..(238): Xaa can be any naturally occurring amino acid
  misc_feature (274)..(274): Xaa can be any naturally occurring amino acid
  misc_feature (299)..(299): Xaa can be any naturally occurring amino acid
  misc_feature (331)..(331): Xaa can be any naturally occurring amino acid
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESARLEAEARLVRESKQLIMTTTNNHPLHLQHHHQLVNINPSTQTHGVVMTKAAATTSSLPCLDVLKAWQGAWSSKPIIRDNCSXNTIVSGMNMMMAMDDLESPTSTLNFPAAYNATYPMXSLNGKQLPQFKEELDNTMVLQEIHXYNGCTDESTTTNAWFAEDGLIRSNGGNDGSSXVILPAAASALSNIMEGFCGTADMLVYNSDEQNIAAGEISDNGGGGGGGGDGSCGRSGSNVEESRSYWDSILNLVNASPSGSPVF

SEQ ID NO: 95 - DNA, 1248 bp, Artificial sequence
  Synthetic polynucleotide
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacwgacaacgagatcaagaactactggaacacgcacctcaagaaragactgaccaagatgggcatcgaccccgtgacccacaagcccaagcaccatgatgcatacggttccggcagcggccacgctaaggacgctgcyaacctgagccacatggcccagtgggagagcgcccgtctcgaagccgaggccaggctsgtcagagaatctaagcagctratcatgaccaccaccaacaatcacccgctccatctccaacaccaccaccagctcgtcaacatcaacccytccacccaaactcayggagtagtcatgaccaaagccgccgccaccacgtcatcgctgccgtgtctggatgtgctgaaagcttggcaaggggcgtggtcgtcaaagcccataattcgtgacaactgyagtartaacacaattgtttcgggtatgaatatgatgatggccatggacgacctcgagtctccaacatccackttgaacttcccggcagcgtacaacgcaacatatccaatgcaatcccttaacggaaagcaattgccccagttcaaagaagagctggacaacaccatggtgctgcargagattcatscatacaacggttgcacggatgaaagtacaactactaatgcgtggttcgcggaggacggtttgattaggtcaaatggtggaaacgatggctcgagcaatgtgattctgccggctgctgcttctgcactttccaatatcatggagggtttctgtggcaccgcggatatgttggtttacaactcggatgagcagaacatagcggccggggagatctcagataatggcggaggaggaggtggaggcggagatggaagctgtgggcgtagtgggagcaacgttgaggagagcaggagctactgggacagtatactgaatttggtgaatgcttctccatccggctcgcctgtgttttaa

SEQ ID NO: 96 - protein, 413 aa, Artificial sequence
  Synthetic polypeptide
  misc_feature (236)..(236): Xaa can be any naturally occurring amino acid
  misc_feature (297)..(297): Xaa can be any naturally occurring amino acid
MGRSPCCDKVGLKKGPWTPEEDQKLLSYIQEYGHGSWRALPAKALQRCGKSCRLRWTNYLRPDIKRGKFSLQEEQTIIQLHALLGNWSAIANHLPKRTDNEIKNYWNTHLKKRLTKMGIDPVTHKPKHHDAYGSGSGHAKDAANLSHMAQWESARLEAEARLVRESKQLIMTTTNNHPLHLQHHHQLVNINPSTQTHGVVMTKAAATTSSLPCLDVLKAWQGAWSSKPIIRDNCSXNTIVSGMNMMMAMDDLESPTSTLNFPAAYNATYPMQSLNGKQLPQFKEELDNTMVLQEIHXYNGCTDESTTTNAWFAEDGLIRSNGGNDGSSNVILPAAASALSNIMEGFCGTADMLVYNSDEQNIAAGEISDNGGGGGGGGDGSCGRSGSNVEESRSYWDSILNLVNASPSGSPVF

SEQ ID NO: 97 - DNA, 1248 bp, Artificial sequence
  Synthetic polynucleotide
atggggaggtctccatgttgtgataaggtgggtttraaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccaygatgcatacggttccggyagcggccacgcyaaggacgctgccaacctsagccacatggcccagtgggagagcgccmgkctsgaagccgaggccaggctggtcagagaatcyaagcagctgatcatgaccaccamcaacaatcaccctctccaactccaactccaacaccaccaccagctcgtcaacatcaacccctccacccaaactcacggagtagtcatgaccaaagccgccgccrccacgycatcgctgccgtgtctggatgtgctgaaagcttggcaaggggcrtggtcgkcaaagcccataattcgtgacaactgtagtagtaacacaattgtttcgggtatgaatatgatkatggccatggacgacctcgagtctccaamatccacgttgaacttcccggcagcgtacaacgcaacatatccaatgcawtmccttaacggaaagcaattkccycagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagmaatgtgattctgccgsctgctgcwgctgcamtttccaatatmatggagggtttctgtggcaccgcggatatgttggtttacaactcggatgagcagaacatagcggccggggagatctcakataatgacggaggcggaggcggagatggaagctgtgggcgtagtggsagcaacgttgaggagarcaggagctactgggacagtatactgaatttggtgaatgcktcwccatccggctcgcctgtgttttra

SEQ ID NO: 98 - protein, 415 aa, Artificial sequence
  Synthetic polypeptide
  misc_feature (157)..(157): Xaa can be any naturally occurring amino acid
  misc_feature (176)..(176): Xaa can be any naturally occurring amino acid
  misc_feature (211)..(211): Xaa can be any naturally occurring amino acid
  misc_feature (213)..(213): Xaa can be any naturally occurring amino acid
  misc_feature (230)..(230): Xaa can be any naturally occurring amino acid
  misc_feature (250)..(250): Xaa can be any naturally occurring amino acid
  misc_feature (260)..(260): Xaa can be any naturally occurring amino acid
  misc_feature (276)..(277): Xaa can be any naturally occurring amino acid
  misc_feature (283)..(283): Xaa can be any naturally occurring amino acid
  misc_feature (332)..(332): Xaa can be any naturally occurring amino acid
  misc_feature (338)..(338): Xaa can be any naturally occurring amino acid
  misc_feature (343)..(343): Xaa can be any naturally occurring amino acid
  misc_feature (373)..(373): Xaa can be any naturally occurring amino acid
  misc_feature (394)..(394): Xaa can be any naturally occurring amino acid
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESAXLEAEARLVRESKQLIMTTXNNHPLQLQLQHHHQLVNINPSTQTHGVVMTKAAAXTXSLPCLDVLKAWQGAWSXKPIIRDNCSSNTIVSGMNMXMAMDDLESPXSTLNFPAAYNATYPMXXLNGKQXPQFKEELDNTMVLQEIHAYNGCTDESTTTHAWFAEDGLIRSNGGNDGSXNVILPXAAAAXSNIMEGFCGTADMLVYNSDEQNIAAGEISXNDGGGGGDGSCGRSGSNVEEXRSYWDSILNLVNASPSGSPVF

SEQ ID NO: 99 - DNA, 1242 bp, Artificial sequence
  Synthetic polynucleotide
atggggaggtctccatgttgtgataaggtgggtttgaaaaaaggaccatggactcctgaggaagaccaaaagcttttgtcttacatccaagagtatggccatggaagctggcgtgctttgcccgcaaaagcagggctccaaagatgtgggaagagctgtcgactgaggtggactaattacctgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacacgcacctcaagaaaagactgaccaagatgggcatcgaccccgtgacccacaagcccaagcaccaygatgcatacggttccggcagcggccacgcyaaggacgctgccaacctsagccacatggcccagtgggagagcgccmgkctsgaagccgaggccaggctggtcagagaatcyaagcagctgatcatgaccaccamcaacaatcaccctctccaactccaactccaacaccaccaccagctcgtcaacatcaacccctccacccaaactcacggagtagtcatgaccaaagccgccgccrccacgycatcgctgccgtgtctggatgtgctgaaagcttggcaaggggcrtggtcgkcaaagcccataattcgtgacaactgtagtagtaacacaattgtttcgggtatgaatatgatkatggccatggacgacctcgagtctccaacatccacgttgaacttcccggcagcrtacaacgcaacatatccaatgcawtmccttaacggaaagcaattkccycagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagmaatgtgattctgccgsctgctgcwgctgcamtttccaatatcatggagggtttctgtggcaccgcggatatgttggtttacaactcggatgagcagaacatagcggccggggagatctcakataatggcggaggcggagatggaagctgtgggcgtagtggsagcaacgttgaggagarcaggagctactgggacagtatactgaatttggtgaatgcktcwccatccggytcgcctgtgttttra

SEQ ID NO: 100 - protein, 413 aa, Artificial sequence
  Synthetic polypeptide
  misc_feature (157)..(157): Xaa can be any naturally occurring amino acid
  misc_feature (176)..(176): Xaa can be any naturally occurring amino acid
  misc_feature (211)..(211): Xaa can be any naturally occurring amino acid
  misc_feature (213)..(213): Xaa can be any naturally occurring amino acid
  misc_feature (230)..(230): Xaa can be any naturally occurring amino acid
  misc_feature (250)..(250): Xaa can be any naturally occurring amino acid
  misc_feature (276)..(277): Xaa can be any naturally occurring amino acid
  misc_feature (283)..(283): Xaa can be any naturally occurring amino acid
  misc_feature (332)..(332): Xaa can be any naturally occurring amino acid
  misc_feature (338)..(338): Xaa can be any naturally occurring amino acid
  misc_feature (343)..(343): Xaa can be any naturally occurring amino acid
  misc_feature (373)..(373): Xaa can be any naturally occurring amino acid
  misc_feature (392)..(392): Xaa can be any naturally occurring amino acid
MGRSPCCDKVGLKKGPWTPEEDQKLLSYIQEYGHGSWRALPAKAGLQRCGKSCRLRWTNYLRPDIKRGKFSLQEEQTIIQLHALLGNRWSAIANHLPKRTDNEIKNYWNTHLKKRLTKMGIDPVTHKPKHHDAYGSGSGHAKDAANLSHMAQWESAXLEAEARLVRESKQLIMTTXNNHPLQLQLQHHHQLVNINPSTQTHGVVMTKAAAXTXSLPCLDVLKAWQGAWSXKPIIRDNCSSNTIVSGMNMXMAMDDLESPTSTLNFPAAYNATYPMXXLNGKQXPQFKEELDNTMVLQEIHAYNGCTDESTTTHAWFAEDGLIRSNGGNDGSXNVILPXAAAAXSNIMEGFCGTADMLVYNSDEQNIAAGEISXNGGGGDGSCGRSGSNVEEXRSYWDSILNLVNASPSGSPVF

SEQ ID NO: 101 - DNA, 1242 bp, Artificial sequence
  Synthetic polynucleotide
atggggaggtctccatgttgtgataaggtgggtttgaaaaaaggaccatggactcctgaggaagaccaaaagcttttgtcttacattgaagagtatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 102 - DNA, 1242 bp, Artificial sequence
  Synthetic polynucleotide
atggggaggtctccatgttgtgataaggtgggtttgaaaaaaggaccatggactcctgaggaagaccaaaagcttttgtcttacatttaagagtatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 103 - DNA, 1235 bp, Artificial sequence
  Synthetic polynucleotide
atggggaggtctccatgttgtgataaggtgggtttgaaaaaaggaccatggactcctgaggaagaccaaaagcttttgtcttacattatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 104 - DNA, 1231 bp, Artificial sequence
  Synthetic polynucleotide
atggggaggtctccatgttgtgataaggtgggtttgaaaaaaggaccatggactcctgaggaagaccaaaagcttttgtcttacatcccatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 105 - DNA, 1229 bp, Artificial sequence
  Synthetic polynucleotide
atggggaggtctccatgttgtgataaggtgggtttgaaaaaaggaccatggactcctgaggaagaccaaaagcttttgtcttacatcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 106 - DNA, 1219 bp, Artificial sequence
  Synthetic polynucleotide
atggggaggtctccatgttgtgataaggtgggtttgaaaaaaggaccatggactcctgaggaagaccaaaagcttttatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcagcgatagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 107 - DNA, 1235 bp, Artificial sequence
  Synthetic polynucleotide
atggggaggtctccatgttgtgataaggtgggtttgaaaaaaggaccatggactcctgaggaagaccaaaagcttttgtcttacatccaagagtatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtagcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 108 - DNA, 1235 bp, Artificial sequence
  Synthetic polynucleotide
atggggaggtctccatgttgtgataaggtgggtttgaaaaaaggaccatggactcctgaggaagaccaaaagcttttgtcttacatccaagagtatgggcatggaagctggcgtgctttgcccgcaaaagcagggctccaaagatgtgggaagagctgtcgactgaggtggactaattacttgaggcctgatatcaaaagaggaaagtttagtttgcaagaagagcagactatcattcaactccatgctcttctcggaaataggtggtcgcaaatcatttgcccaaaaggacagacaacgagatcaagaactactggaacactcacctcaagaaaagactgaccaagatgggcatcgaccccgtgacccacaagcccaagcaccacgatgcatacggttccggcagcggccacgccaaggacgctgccaacctcagccacatggcccagtgggagagcgcccggctcgaagccgaggccaggctggtcagagaatccaagcagctgatcatgaccaccaacaacaatcaccctctccaactccaactccaacaccaccaccagctcgtcaacatcaacccctccacccaaactcacggagtagtcatgaccaaagccgccgccgccacgtcatcgctgccgtgtctggatgtgctgaaagcttggcaaggggcatggtcgtcaaagcccataattcgtgacaactgtagtagtaacacaattgtttcgggtatgaatatgatgatggccatggacgacctcgagtctccaacatccacgttgaacttcccggcagcgtacaacgcaacatatccaatgcaatcccttaacggaaagcaattgcctcagttcaaagaagagctggacaacaccatggtgctgcaggagattcatgcatacaacggttgcacggatgaaagtacaactactcatgcgtggttcgcggaggacggtttgattaggtcaaatggtggaaacgatggctcgagcaatgtgattctgccggctgctgcagctgcactttccaatatcatggagggtttctgtggcaccgcggatatgttggtttacaactcggatgagcagaacatagcggccggggagatctcagataatggcggaggcggagatggaagctgtgggcgtagtggcagcaacgttgaggagaacaggagctactgggacagtatactgaatttggtgaatgcctcaccatccggctcgcctgtgttttaa

SEQ ID NO: 109 - DNA, 20 bp, Artificial sequence
  Synthetic oligonucleotide
tggtcttcctcaggagtcca

SEQ ID NO: 110 - DNA, 23 bp, Artificial sequence
  Synthetic oligonucleotide
tcttacatccaagagtatgggca

SEQ ID NO: 111 - DNA, 20 bp, Artificial sequence
  Synthetic oligonucleotide
tacatccaagagtatgggca

SEQ ID NO: 112 - DNA, 20 bp, Artificial sequence
  Synthetic oligonucleotide
atatcaggcctcaagtaatt

SEQ ID NO: 113 - DNA, 20 bp, Artificial sequence
  Synthetic oligonucleotide
ctcttttgatatcaggcctc

SEQ ID NO: 114 - DNA, 20 bp, Artificial sequence
  Synthetic oligonucleotide
gtttgcaagaagagcagact

SEQ ID NO: 115 - DNA, 20 bp, Artificial sequence
  Synthetic oligonucleotide
caagaagagcagactatcat

SEQ ID NO: 116 - DNA, 20 bp, Artificial sequence
  Synthetic oligonucleotide
attcaactccatgctcttct

SEQ ID NO: 117 - DNA, 20 bp, Artificial sequence
  Synthetic oligonucleotide
cgagaagagcatggagttga

SEQ ID NO: 118 - DNA, 11 bp, Artificial sequence
  Synthetic oligonucleotide
aagagtatggg

SEQ ID NO: 119 - DNA, 13 bp, Artificial sequence
  Synthetic oligonucleotide
ccaagagtatggg

SEQ ID NO: 120 - DNA, 23 bp, Artificial sequence
  Synthetic oligonucleotide
gtcttacatccaagagtatgggc

SEQ ID NO: 121 - DNA, 1251 bp, Fragaria vesca
atggggaggtctccatgttgcgagaaggtgggtttgaaaaaaggaccatggacgcctgaggaagaccagaagctcctgtcttacatccaggaatacggccatggaagctggcgtgctttgccggctaaagcagggctccaaagatgtgggaagagctgccgactgaggtggactaactacctgaggcctgatatcaaaagaggaaagtttagtttgcaggaagagcaaaccatcattcaactccatgctcttctgggaaataggtggtcagctatagcaaatcacttacccaaaaggacagacaacgagatcaagaactactggaacacacacctcaagaaaagactgaccaagatgggcatcgaccccgtgacacacaagcccaagcaccacgaagcctacgggtccggcagcggccacggcaaggacgctgccaacctgagccacatggctcagtgggagagcgctcggctcgaggccgaggccaggctggtcagagaatccaagcagctcatcatgatcaatcaccctctccatggccagcttcagcaccatcagctcctcaacatcagcccctccacccaaactcacggacttcacatgagcaaagccgccgccgccacgtcagcgctgccgtgtctggatgtgctcaaagcctggcaaggggcgtggtcgtcaaagccgataatccgagacaacaacggtggtaataataacacgattgtttcgggtatgaatttgatgatggccatggacgacctcgagtctccgacgtccacgttgagcttcccggctgctgcgtacaacaacgcagcatatccaatgcagtctcttaacgccaataagcaactatctgacttcaaacaagagctgcaccactcgatggtgctgcaagacattcatgcatacaatggttgtacggatgcaagtacaacaactaccaacccgtggttcgctgacgacggtatgattaggtcaagtggcgaaaacgatgacgtgagcaacgtcatgctgccggccgctgctgctgcgttttccaatatcatggagggtttctgcggcggagccgccacctcggacatgttggtttacaactccgatgatcagaacatgacggccggagagatctctgatacaggcggggatggaagctgtgggcgtagtggcaacaacattgaggagaacaggagctactgggacagtatactcaacttggtgaatgcttcaccatccgggtcgcctgtgttttaa

SEQ ID NO: 122 - DNA, 20 bp, Artificial sequence
  Synthetic oligonucleotide
cgctctcccactgggccatg

SEQ ID NO: 123 - DNA, 20 bp, Artificial sequence
  Synthetic oligonucleotide
caggaagagcaaaccatcat

SEQ ID NO: 124 - DNA, 23 bp, Artificial sequence
  Synthetic oligonucleotide
gccggcaaagcacgccagcttcc

SEQ ID NO: 125 - DNA, 20 bp, Artificial sequence
  Synthetic oligonucleotide
gtttgcaggaagagcaaacc

SEQ ID NO: 126 - DNA, 20 bp, Artificial sequence
  Synthetic oligonucleotide
ccagaagagcatggagttga

SEQ ID NO: 127 - DNA, 23 bp, Artificial sequence
  Synthetic oligonucleotide
atatcaggcctcaggtagttagt

SEQ ID NO: 128 - DNA, 23 bp, Artificial sequence
  Synthetic oligonucleotide
aaacccaccttctcgcaacatgg

SEQ ID NO: 129 - DNA, 20 bp, Artificial sequence
  Synthetic oligonucleotide
ctcttcctgcaaactaaact

SEQ ID NO: 130 - DNA, 20 bp, Artificial sequence
  Synthetic oligonucleotide
aaaaaaggaccatggacgcc

(19) United States
(12) Patent Application Publication
(10) Pub. No.: US 2023/0063560 A1
(43) Pub. Date: Mar. 2, 2023

(54) THORNLESS AND/OR PRICKLELESS RUBUS PLANTS

(71) Applicant: Pairwise Plans Services, Inc., Durham, NC (US)

(72) Inventor: Brian Charles Wilding Crawford, Cary, NC (US)

(21) Appl. No.: 17/795,018

(22) PCT Filed: Feb. 4, 2021
(86) PCT No.: PCT/US2021/016628

§ 371 Date: Jul. 25, 2022

Related U.S. Application Data
(60) Provisional application No. 62/970,024, filed on Feb. 4, 2020.

(51) Int. Cl. C12N 15/82; C07K 14/415

(52) U.S. Cl. CPC C12N 15/8262; C07K 14/415

(57) ABSTRACT

This invention relates to compositions and methods for modifying MIXTA transcription factors, including MIXTA-Like transcription factors, in Rubus plants to reduce or eliminate thorns and prickles in Rubus plants. The invention further relates to Rubus plants produced using the methods and compositions of the invention.

### STATEMENT REGARDING ELECTRONIC FILING OF A SEQUENCE LISTING

A Sequence Listing in ASCII text format, submitted under 37 C.F.R. § 1.821, entitled 1499.18.WO_ST25.txt, 368,292 bytes in size, generated on Feb. 3, 2021 and filed via EFS-Web, is provided in lieu of a paper copy. This Sequence Listing is hereby incorporated herein by reference into the specification for its disclosures.

### STATEMENT OF PRIORITY

This application claims the benefit, under 35 U.S.C. § 119 (e), of U.S. Provisional Application No. 62/970,024 filed on Feb. 4, 2020, the entire contents of which is incorporated by reference herein.

### FIELD OF THE INVENTION

This invention relates to compositions and methods for modifying MIXTA transcription factors, including MIXTA-Like transcription factors, in Rubus plants to reduce or eliminate thorns and prickles in Rubus plants. The invention further relates to Rubus plants produced using the methods and compositions of the invention.

### BACKGROUND OF THE INVENTION

Trichomes are hair-like structures of epidermal origin which can protect plants against damage by insects, pathogens, UV irradiation, low temperature and excessive transpiration. In addition, trichomes may help plants attract pollinators and disperse seeds. Trichomes exhibit high morphological variation, and can be divided into several forms, which could be unicellular or multicellular, glandular or non-glandular, and branched or unbranched. Trichomes are easily observed and their loss can be detrimental to the plant. The study of trichome development was explored when molecular genetics was applied to plants. Indeed, as trichomes can be observed at the earliest stages of development they have provided a good phenotypic marker for studying tool development for gene editing in Arabidopsis.

The canes of wild Blackberry species and other Rubus species are covered from the bottom to the top with differing densities of thorns. These thorns are not derived from the epidermis rather from the vascular tissue and are more correctly referred to as prickles or spines. Thorns (e.g., prickles or spines) interfere with plant propagation, plant care and fruit harvesting. Additionally, the presence of thorns can be a liability if a consumer were to inadvertently eat a thorn.

Today, there are four sources of thornlessness available to plant breeders. One is the recessive gene ‘s’ used by the John Innes program in the United Kingdom to develop ‘Merton Thornless’, which is still the source of thornlessness in modern tetraploid cultivars. The use of ‘Merton Thornless’ has been very important in advancing thornlessness, particularly in erect and semi-erect genotypes. Gene ‘s’ is a recessive trait. Two advantages of this gene are the complete absence of thorns and the ability to screen seedlings for the absence of glandular trichomes. The disadvantages include the 35:1 segregation ratio in tetraploids and the linkage of this trait to undesirable traits such as lack of winter hardiness, acid fruit and later harvest season.

Another source of thornlessness is the dominant gene ‘Sf’ from ‘Austin Thornless’. The advantage of ‘Sf’ is the genetic dominance, which has provided a thornlessness source at the 6× and higher ploidy levels (‘Austin Thornless’ is an octoploid). ‘Sf’ has been important in breeding trailing types. However, several detrimental traits are linked to this locus, including a trailing growth habit and susceptibility to downy mildew. Also, plants derived from this source of thornlessness can have thorns on the basal 0.3 m of the cane; these same canes are thornless beyond this point and are commercially thornless since fruit is borne only in the thornless area of the cane. Therefore, the identification of thornless progeny using this source of thornlessness cannot be fully done until seedlings are 20-30 cm tall.

The dominant ‘Sfte’ locus is another source of thornlessness and is from non-chimeras of ‘Thornless Evergreen’. This locus is not used in breeding as it reverts regularly in the field and also is associated with a lot of undesirable traits. A fourth source of thornlessness available for breeding includes, the semi-dominant ‘Sfl’ gene of ‘Lincoln Logan’. This allele was originally identified as a chimera of ‘Loganberry’. A tissue culture technique in which a ‘Loganberry’-type clone (L654) was used resulted in a spontaneous embryo from callus tissue. ‘Lincoln Logan’ was released from this effort and was then used in New Zealand and USDA-ARS Oregon breeding programs. The first cultivars with the ‘Sfl’ source are likely to be released soon. However, there are extensive fertility problems and also it appears that two separate loci may be required to produce ‘Sfl’ thornlessness.

Blackberry varieties are clonally propagated. Currently, if breeders have a thorny blackberry variety it is not possible to remove the thorns without laborious crossing and backcrossing to introgress one of the four mentioned thornless alleles. Making a blackberry plant thornless in one generation would be a dramatic advance for blackberry breeding. Accordingly, new sources for use in generating thornless and prickleless Rubus plants would be advantageous.

### SUMMARY OF THE INVENTION

One aspect of the invention provides a Rubus plant or plant part thereof comprising at least one non-natural mutation in at least one copy of an endogenous gene encoding a MIXTA transcription factor.

One aspect of the invention provides a Rubus plant cell, comprising an editing system comprising: (a) a CRISPR-associated effector protein; and (b) a guide nucleic acid having a spacer sequence with complementarity to an endogenous target gene encoding a wild type MIXTA transcription factor.

A second aspect of the invention provides a Rubus plant cell comprising at least one non-naturally occurring mutation within a MIXTA transcription factor gene, wherein the mutation is a substitution, insertion or a deletion that is introduced using an editing system that comprises a nucleic acid binding domain that binds to a target site in the MIXTA transcription factor gene, wherein the MIXTA transcription factor gene comprises a nucleotide sequence of any one of SEQ ID NOs:63-70, 77, 84-89, 93, 95, 97, or 99, or a sequence having at least 95% identity to the nucleotide sequence of SEQ ID NOs:78 or SEQ ID NO:83, or encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57.

A third aspect provides a Rubus plant cell, comprising an editing system comprising: (a) a CRISPR-associated effector protein; and (b) a guide nucleic acid having a spacer sequence with complementarity to an endogenous target gene encoding a wild type MIXTA transcription factor.

A fourth aspect of the invention provides a method of producing/breeding a transgene-free edited Rubus plant, comprising: crossing the Rubus plant of any one of the preceding claims with a transgene free Rubus plant, thereby introducing the at least one non-natural mutation or the modification into the Rubus plant that is transgene-free; and selecting a progeny Rubus plant that comprises the at least one non-natural mutation or the modification and is transgene-free, thereby producing a transgene free edited Rubus plant.

A fifth aspect of the invention provides a method for editing a specific site in the genome of a Rubus plant cell, the method comprising cleaving, in a site specific manner, a target site within an endogenous MIXTA transcription factor gene in the Rubus plant cell, the endogenous MIXTA transcription factor gene comprising the nucleotide sequence of SEQ ID NOs: 63-70, 77, 84-89, 93, 95, 97, or 99 or a sequence having at least 95% identity to the nucleotide sequence of SEQ ID NO:78 or SEQ ID NO:83, or encoding a polypeptide comprising the sequence of any one of the amino acid sequences of SEQ ID NOs:57-62, 71-76, 90-92, 94, 96, 98, or 100, or a sequence having at least at least 94% identity to any one of the amino acid sequences of SEQ ID NO:57, thereby generating an edit in the endogenous MIXTA transcription factor gene of the Rubus plant cell and producing a plant cell comprising the edit in the endogenous MIXTA transcription factor gene.

A sixth aspect of the invention provides a method for making a Rubus plant, comprising: (a) contacting a population of Rubus plant cells comprising at least one wild type endogenous MIXTA transcription factor gene with a nuclease linked to a DNA binding domain (e.g., an editing system) that binds to a target site in the at least one wild type endogenous MIXTA transcription factor gene, wherein the at least one wild type endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of 63-70, 77, 84-89, 93, 95, 97, or 99 or a sequence having at least 95% identity to the nucleotide sequence of SEQ ID NO:78 or SEQ ID NO:83; (b) selecting a Rubus plant cell from said population that comprises a mutation in the at least one wild type endogenous MIXTA transcription factor gene, and (c) growing the selected Rubus plant cell into a Rubus plant comprising the MIXTA transcription factor mutation.

A seventh aspect of the invention provides a method for reducing thorns and prickles in a Rubus plant or part thereof, comprising (a) contacting a Rubus plant cell comprising a wild type endogenous MIXTA transcription factor gene with a nuclease targeting the wild type endogenous MIXTA transcription factor gene, wherein the nuclease is linked to a DNA binding domain that binds to a target site in the wild type endogenous MIXTA transcription factor gene, wherein the wild type endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83; (b) growing the plant cell into a plant, thereby reducing thorns and prickles in the Rubus plant, thereby reducing thorns and prickles in the Rubus plant or part thereof.

An eighth aspect provides a method for producing a Rubus plant or part thereof comprising at least one cell having a mutated endogenous MIXTA transcription factor gene, the method comprising contacting a target site in an endogenous MIXTA transcription factor gene in the Rubus plant or plant part with a nuclease comprising a cleavage domain and a DNA-binding domain, wherein the DNA binding domain binds to a target site in the endogenous MIXTA transcription factor gene, wherein the endogenous MIXTA transcription factor gene (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO: 78 or SEQ ID NO:83, thereby producing the Rubus plant or part thereof comprising at least one cell having a mutation in the endogenous MIXTA transcription factor gene.

An ninth aspect provides a method for producing a Rubus plant or part thereof comprising a mutated endogenous MIXTA transcription factor and reduced thorns and/or prickles, the method comprising contacting a target site in an endogenous MIXTA transcription factor gene in the Rubus plant or plant part with a nuclease comprising a cleavage domain and a DNA-binding domain, wherein the DNA binding domain binds to a target site in the endogenous MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83; thereby producing the Rubus plant or part thereof comprising a mutated endogenous MIXTA transcription factor and reduced thorns and/or prickles.

A tenth aspect provides a method for reducing thorns and/or prickles in a Rubus plant or part thereof, comprising (a) contacting a plant cell comprising a wild type endogenous MIXTA transcription factor gene with a nuclease targeted to the wild type endogenous MIXTA transcription factor gene, wherein the nuclease is linked to a DNA binding domain that binds to a target site in the endogenous MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83; thereby producing a Rubus plant or part thereof having reduced thorns and/or prickles.

An eleventh aspect provides method for producing a plant or part thereof comprising at least one cell having a mutated endogenous MIXTA transcription factor gene, the method comprising contacting a target site in an endogenous MIXTA transcription factor gene in the plant or plant part with a nuclease comprising a cleavage domain and a DNA-binding domain, wherein the DNA binding domain binds to the target site in the MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83; thereby producing a plant or part thereof comprising at least one cell having a mutated endogenous MIXTA transcription factor gene.

A twelfth aspect provides a method for producing a plant or part thereof comprising at least one cell having a mutated endogenous MIXTA transcription factor gene, the method comprising contacting a target site in an endogenous MIXTA transcription factor gene in the plant or plant part with a nuclease comprising a cleavage domain and a DNA-binding domain, wherein the DNA binding domain binds to the target site in the MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78; thereby producing a plant or part thereof comprising at least one cell having a mutated endogenous MIXTA transcription factor gene. In some aspects, the mutated endogenous MIXTA transcription factor gene is a null allele that results in a plant having reduced thorns and/or prickles. In some aspects the mutated endogenous MIXTA transcription factor gene comprises a dominant negative mutation that results in a plant having reduced thorns and/or prickles.

A thirteenth aspect provides a guide nucleic acid (e.g., gRNA, gDNA, crRNA, crDNA) that binds to a target site in a MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83.

A fourteenth aspect provides, a system is provided comprising the guide nucleic acid and a CRISPR-Cas effector protein that associates with the guide nucleic acid.

In a fifteenth aspect, a gene editing system is provided comprising a CRISPR-Cas effector protein in association with a guide nucleic acid, wherein the guide nucleic acid comprises a spacer sequence that binds to a MIXTA transcription factor gene.

In another aspect, a complex comprising a CRISPR-Cas effector protein is provided comprising a cleavage domain and a guide nucleic acid, wherein the guide nucleic acid binds to a target site in a MIXTA transcription factor gene, wherein the MIXTA transcription factor gene comprises a nucleotide sequence of any one of SEQ ID NOs:63-70, 77, 84-89, 93, 95, 97, or 99 or a sequence having at least 95% identity to the nucleotide sequence of SEQ ID NO: 78 or SEQ ID NO:83, or encodes a encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57, wherein the cleavage domain cleaves a target strand in the MIXTA transcription factor gene.

In a sixteenth aspect, an expression cassette is provided comprising (a) a polynucleotide encoding CRISPR-Cas effector protein comprising a cleavage domain and (b) a guide nucleic acid that binds to a target site in an endogenous MIXTA transcription factor gene, wherein the guide nucleic acid comprises a spacer sequence that is complementary to and binds: (i) to a portion of the nucleotide sequence of any one of SEQ ID NOs:77 or 84-89 or a nucleotide sequence having at least 95% identity to the nucleotide sequence of SEQ ID NO:78 or SEQ ID NO:83 or (ii) to a portion of a nucleotide sequence encoding any one of the amino acid sequences of SEQ ID NOs:73-76 or 90-92.

A seventeenth aspect of the invention provides a nucleic acid encoding a null allele and/or a dominant negative mutation of a MIXTA transcription factor.

In an eighteenth aspect, a Rubus plant or part thereof is provided comprising a nucleic acid of the invention.

In a further aspect, a Rubus plant or part thereof is provided comprising reduced thorns and/or prickles.

Further provided are plants comprising in their genome one or more mutated MIXTA transcription factors that have reduced ability to bind to DNA that are produced by the methods of the invention as well as polypeptides, polynucleotides, nucleic acid constructs, expression cassettes and vectors for making a plant of this invention. Additionally provided are plants comprising in their genome one or more mutated MIXTA transcription factors and one or more mutations in an endogenous polynucleotide of interest, including in at least one AG clade MADS-box transcription factor gene, including but not limited to SEEDSTICK (STK) genes.

These and other aspects of the invention are set forth in more detail in the description of the invention below.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron.

SEQ ID NOs:23-29 are exemplary cytosine deaminase sequences useful with this invention.

SEQ ID NOs:30-34 are exemplary adenine deaminase amino acid sequences useful with this invention.

SEQ ID NO:35 is an exemplary uracil-DNA glycosylase inhibitor (UGI) sequences useful with this invention.

SEQ ID NOs:36-38 provides an example of a protospacer adjacent motif position for a Type V CRISPR-Cas12a nuclease.

SEQ ID NOs:39-41 provide example peptide tags and affinity polypeptides useful with this invention.

SEQ ID NOs:42-52 provide example RNA recruiting motifs and corresponding affinity polypeptides useful with this invention.

SEQ ID NOs:53-56, 109-117 and 122-130 are example spacer sequences for nucleic acid guides useful with this invention.

SEQ ID NOs:57-62 are MIXTA transcription factor polypeptide sequences from blackberry.

SEQ ID NOs:63-68 are MIXTA transcription factor polynucleotide sequences from blackberry.

SEQ ID NOs:69-70 are MIXTA transcription factor polynucleotide sequences from black raspberry and red raspberry, respectively.

SEQ ID NOs:71-72 are MIXTA transcription factor polypeptide sequences from black raspberry and red raspberry, respectively.

SEQ ID NO:73 shows a portion of the MIXTA transcription factor polypeptide sequences (amino acid residues 97-114 of any one of SEQ ID NOs:57-62 or 71-72).

SEQ ID NO:74 shows a portion of the MIXTA transcription factor polypeptide sequences (amino acid residues 90-125 of any one of SEQ ID NOs:57-62 or 71-72).

SEQ ID NO:75 shows a portion of the MIXTA transcription factor polypeptide sequences (amino acid residues 77-121 of any one of SEQ ID NOs:57-62 or 71-72).

SEQ ID NO:76 shows a portion of the MIXTA transcription factor polypeptide sequences (amino acid residues 77-125 of any one of SEQ ID NOs:57-62 or 71-72).

SEQ ID NO:77 shows a portion of the MIXTA transcription factor polynucleotide sequences (nucleotides 250-329 of any one of SEQ ID NOs:63-70).

SEQ ID NO:78 shows a portion of a blackberry MIXTA transcription factor polynucleotide sequence (nucleotide bases 1-390 of SEQ ID NO:63).

SEQ ID NOs:79-80 are exemplary Cas9 sequences useful with this invention.

SEQ ID NOs:81-82 are the portions of a MIXTA polypeptide and corresponding amino acid sequence shown in FIG. 3.

SEQ ID NO:83 shows a portion of the MIXTA transcription factor polynucleotide sequences (nucleotide residues 1-390 of any one of SEQ ID NOs:64, 66, 67, 68, 70, 93, 95, 97, or 99.

SEQ ID NO:84 shows a portion of the MIXTA transcription factor polynucleotide sequences (nucleotide residues 42-122 of any one of SEQ ID NOs:63-68, 70, 93, 95, 97, or 99.

SEQ ID NO:85 shows a portion of a MIXTA transcription factor polynucleotide sequence (nucleotide residues 42-122 of any one of SEQ ID NO:69.

SEQ ID NO:86 shows a portion of the MIXTA transcription factor polynucleotide sequences (nucleotide residues 42-98 of any one of SEQ ID NOs:63-70, 93, 95, 97, or 99.

SEQ ID NO:87 shows a portion of the MIXTA transcription factor polynucleotide sequences (nucleotide residues 57-109 of any one of SEQ ID NOs:63-70, 93, 95, 97, or 99.

SEQ ID NO:88 shows a portion of the MIXTA transcription factor polynucleotide sequences (nucleotide residues 69-97 of any one of SEQ ID NOs:63-70, 93, 95, 97, or 99.

SEQ ID NO:89 shows a portion of the MIXTA transcription factor polynucleotide sequences (nucleotide residues 259-288 of any one of SEQ ID NOs:63-70, 93, 95, 97, or 99.

SEQ ID NO:90 shows a portion of the MIXTA transcription factor polypeptide sequences (amino acid residues 17-44 of any one of SEQ ID NOs:57-62, 71, 72, 94, 96, 98, or 100).

SEQ ID NO:91 shows a portion of the MIXTA transcription factor polypeptide sequences (amino acid residues 24-36 of any one of SEQ ID NOs:57-62, 71, 72, 94, 96, 98, or 100).

SEQ ID NO:92 shows a portion of the MIXTA transcription factor polypeptide sequences (amino acid residues 87-96 of any one of SEQ ID NOs:57-62, 71, 72, 94, 96, 98, or 100).

SEQ ID NOs:93, 95, 97 and 99 are example consensus sequences for MIXTA transcription factor polynucleotides from two different blackberry lines.

SEQ ID NOs:94, 96, 98, and 100 are the polypeptide sequences that correspond to the for MIXTA transcription factor polynucleotides of SEQ ID NOs:93, 95, 97 and 99. respectively.

SEQ ID NOs:101, 102, 103, 104, 105, 106, 107 and 108 are example edits in endogenous blackberry MIXTA genes.

SEQ ID NOs:118, 119, and 120 are deleted portions from example edited blackberry MIXTA genes as shown in Table 4.

SEQ ID NO:121 is a MIXTA transcription factor polynucleotide sequence from Fragaria vesca.

### BRIEF DESCRIPTION OF THE DRAWINGS

FIG. 1A-1B provide an alignment between the amino acid sequences of SEQ ID NOs:57-62, 71 and 72.

FIG. 2A-2D provide an alignment between the nucleotide sequences of SEQ ID NOs:63-70.

FIG. 3 shows exemplary spacer sequences aligned with the consensus Rubus Exon2-Exon3 junction.

FIG. 4 provides an alignment between portions of edited blackberry MIXTA nucleic acids in blackberry and a portion of the endogenous wild type blackberry MIXTA sequence. From top to bottom: SEQ ID NO:65 (nucleotides 70-151; wild type blackberry MIXTA), SEQ ID NO:101 (nucleotides 70-151; edited blackberry EPS4812 and EPS4366), SEQ ID NO:102 (nucleotides 70-151; edited blackberry EPS4313), SEQ ID NO:103 (nucleotides 70-144; edited blackberry EPS4309A), SEQ ID NO:104 (nucleotides 70-140; edited blackberry EPS4309B), SEQ ID NO:105 (nucleotides 70-138; edited blackberry EPS4309C), SEQ ID NO:106 (nucleotides 70-128; edited blackberry EPS4309D).

FIG. 5 provides an alignment between portions of edited MIXTA nucleic acids in blackberry and a portion of endogenous wild type MIXTA sequence. Nucleic acid sequences from top to bottom: SEQ ID NO:65 (nucleotides 249-301; wild type blackberry MIXTA), SEQ ID NO:107 (nucleotides 249-294; edited blackberry EPS4472), SEQ ID NO:108 (nucleotides 249-294; edited blackberry EPS4281).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount. For example, “reduced thornlessness” or “reduced pricklelessness” can mean a reduction in the production of thorns and/or prickles by about 5% to about 100% as compared to a control plant. Thus, in some aspects, a reduction in the production of thorns and/or prickles may result in no or essentially no thorns and/or prickles.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type endogenous MIXTA transcription factor gene” is an MIXTA transcription factor gene that is naturally occurring in or endogenous to the reference organism, e.g. a Rubus spp.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker- assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

The terms “thorn” and “prickle” (and grammatical variations thereof) will be used interchangeably herein. Thus, while in blackberry, “thorns” are more accurately referred to as “prickles” or “spines,” they may be referred to herein as “thorns” or “prickles” with the absence of thorns or prickles being described as “thornless,” “thornlessness,” “prickleless” or “pricklessness.”

“Substantially thornless” or “substantially prickleless” as used herein refers to having a reduction in the amount of thorns or prickles by about 50%, 51%, 52%, 53%, 54%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5%.

“MIXTA transcription factors” and “MIXTA-like transcription factors” are myeloblastosis (MYB) proteins that are involved in the regulation of plant development, including trichome development. As used herein, the term “MIXTA transcription factor” or “MIXTA transcription factor gene” refers to both “MIXTA” and “MIXTA-like” transcription factors and the genes that encode them.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild Type CRISR-Cas repeat, e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like). In some embodiments, a nucleic acid fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, 550, 600, 650, 700, 750, 800, 850, 900, or 950 or more consecutive nucleotides or any range or value therein of a nucleic acid encoding an MIXTA transcription factor, optionally a MIXTA fragment may be about 50 nucleotides to about 300 nucleotides in length, about 50 nucleotides to about 350 nucleotides in length, about 50 nucleotides to about 400 nucleotides in length, about 50 nucleotides to about 450 nucleotides in length, about 50 nucleotides to about 500 nucleotides in length, about 50 nucleotides to about 600 nucleotides in length, about 50 nucleotides to about 800 nucleotides in length, about 50 nucleotides to about 900 nucleotides in length, about 50 nucleotides to about 950 nucleotides in length, about 100 nucleotides to about 300 nucleotides in length, about 100 nucleotides to about 350 nucleotides in length, about 100 nucleotides to about 400 nucleotides in length, about 100 nucleotides to about 450 nucleotides in length, about 100 nucleotides to about 500 nucleotides in length, about 100 nucleotides to about 600 nucleotides in length, about 100 nucleotides to about 800 nucleotides in length, about 100 nucleotides to about 900 nucleotides in length, or about 100 nucleotides to about 950 nucleotides in length, or any range or value therein. In some embodiments, a nucleic acid fragment of a MIXTA transcription factor gene may be the result of a deletion of nucleotides from the 3′ end, the 5′ end, and/or from within the gene encoding the MIXTA transcription factor. In some embodiments, a deletion of a portion of a gene encoding a MIXTA transcription factor may comprise deletion of a portion of consecutive nucleotides from the 5′ end, the 3′ end, or from within, for example, any one of the nucleotide sequences of SEQ ID NOs:63-70, 93, 95, 97, or 99. In some embodiments, a deletion of a portion of a MIXTA transcription factor gene may comprise deletion of a portion of consecutive nucleotides from of any one of the nucleotide sequences of SEQ ID NOs:63-70, 93, 95, 97, or 99 (e.g., from the 3′ end or 5′ end). In some embodiments, a deletion of a portion of a MIXTA transcription factor gene may comprise a deletion of a portion of consecutive nucleotides from any one of the nucleotide sequences of SEQ ID NOs:63-70, 93, 95, 97, or 99 (e.g., from the 3′ end or 5′ end) of from about 5, 6, 7, 8, 9, 10, 11, 12, 13, 14, 15, 16, 17, 18, 19, 20, 25, 30, 35, 40, 45, or 50 consecutive nucleotides to about 1250 consecutive nucleotides or more (e.g., about 5, 6, 7, 8, 9, 10, 11, 12, 13, 14, 15, 16, 17, 18, 19, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350, 400, 450, 500, 550, 600, 650, 700, 750, 800, 850, 900, 950, 1000, 1100, 1200, 1210, 1220, 1230, 1240 or 1250 or more consecutive nucleotides, or any range or value therein). In some embodiments, a deletion of a portion of a MIXTA transcription factor gene may comprise a deletion of a portion of consecutive nucleotides from the 5′ end of any one of the nucleotide sequences of SEQ ID NOs:63-70, 93, 95, 97, or 99 of about 1 to about 25 consecutive nucleotides (e.g., 1, 2, 3, 4, 5, 6, 7, 8, 9, 10, 11, 12, 13, 14, 15, 16, 17, 18, 19, 20, 21, 22, 23, 24, or 25 consecutive nucleotides (bp)) and the deletion results in a premature stop codon and a truncated protein. In some embodiments, the deletion is a 7 bp, 11bp, 13 bp or 23 bp deletion that results in a premature stop codon and a truncated protein. In some embodiments, deletions as described herein may result in a null allele, which when comprised in a plant can result in a reduced amount of or elimination of thorns or prickles in the plant. In some embodiments, such a deletion may be a dominant negative mutation, which when comprised in a plant can result in a reduced amount of or elimination of thorns or prickles in the plant.

In some embodiments, a “sequence-specific DNA binding domain” may bind to one or more fragments or portions of nucleotide sequences encoding MIXTA transcription factors as described herein.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400 or more consecutive amino acids of a reference polypeptide. In some embodiments, a polypeptide fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 110, 115, 120, 125, 130, 140, or 150 consecutive amino acid residues (or any range or value therein) of a MIXTA transcription factor (e.g., a fragment or a portion of any one of SEQ ID NOs:57-62, 71, 72, 94, 96, 98, or 100.

In some embodiments, a “portion” may be related to the number of amino acids that are deleted from a polypeptide. Thus, for example, a deleted “portion” of an MIXTA transcription factor may comprise at least one amino acid residue (e.g., at least 1, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 320, 330, 340, 350, 360, 370, 380, 390 or 400 or more consecutive amino acid residues and any range or value therein) deleted from any one of the amino acid sequences of SEQ ID NOs:57-62, 71, 72, 94, 96, 98, or 100. In some embodiments, a deletion of a portion of a MIXTA transcription factor may comprise a deletion of a portion of consecutive amino acid residues from the N- or C-terminus of or within any one of the amino acid sequences of SEQ ID NOs:57-62, 71, 72, 94, 96, 98, or 100. In some embodiments, a deletion of a portion of a MIXTA transcription factor may comprise a deletion of a portion of consecutive amino acid residues from the C-terminus of any one of the amino acid sequences of SEQ ID NOs:57-62, 71, 72, 94, 96, 98, or 100. In some embodiments, a deletion of a portion of a MIXTA transcription factor may comprise a deletion of a portion of consecutive amino acid residues from the C-terminus of any one of the amino acid sequences of SEQ ID NOs:57-62, 71, 72, 94, 96, 98, or 100 of from about 10 consecutive amino acids to about 250, about 260, about 270, about 280, about 290, about 300, about 310, about 320, about 330, about 340, about 350, about 380, about 390 or about 400 or more consecutive amino acids, about 20 consecutive amino acids to about 250, about 260, about 270, about 280, about 290, about 300, about 310 about 320, about 330, about 340, about 350, about 380, about 390 or about 400 consecutive amino acids, about 30 consecutive amino acids to about 250, about 260, about 270, about 280, about 290, about 300, about 310, about 320 about 320, about 330, about 340, about 350, about 380, about 390 or about 400 consecutive amino acids. In some embodiments, such a deletion may be a null allele, which when comprised in a Rubus plant can result in a reduced amount of or elimination of thorns or prickles in the plant. In some embodiments, such a deletion may be a dominant negative mutation, which when comprised in a Rubus plant can result in a reduced amount of or elimination of thorns or prickles in the plant.

In some embodiments, a “sequence-specific DNA binding domain” may bind to one or more fragments or portions of nucleotide sequences encoding MIXTA transcription factors as described herein.

As used herein with respect to nucleic acids, the term “functional fragment” refers to nucleic acid that encodes a functional fragment of a polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70 or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more MIXTA transcription factors may be identical or substantially identical (e.g., at least 70% to 99.9% identical, e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% identical or any range or value therein) to one another over at least about 18 consecutive amino acid residues (e.g., SEQ ID NO:73), over at least about 36 consecutive amino acid residues (e.g., SEQ ID NO:74), over at least about 45 consecutive amino acid residues (e.g., SEQ ID NO:75), at least about 49 consecutive amino acid residues (e.g., SEQ ID NO:76), over at least about 28 consecutive amino acid residues (e.g., SEQ ID NO:90), at least about 13 consecutive amino acid residues (e.g., SEQ ID NO:91), at least about 10 consecutive amino acid residues (e.g., SEQ ID NO:92), and the like. In some embodiments, two or more MIXTA transcription factors may be substantially identical across consecutive amino acid residues 1 to about 225, 230, 240, 250, 260, 270, 280, 290, 300, or 310 of the amino acid sequence of SEQ ID NO:57. In some embodiments, two or more MIXTA transcription factors may be substantially identical across consecutive amino acid residue number 1 to about 40, 50, 60, 70, 80, 90, 100, 110, 120, 130, or 140 of the amino acid sequence of SEQ ID NOs:94, 96, 98 or 100.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

A polynucleotide and/or recombinant nucleic acid construct of this invention (e.g., expression cassettes and/or vectors) may be codon optimized for expression. In some embodiments, the polynucleotides, nucleic acid constructs, expression cassettes, and/or vectors of the editing systems of the invention (e.g., comprising/encoding a sequence-specific DNA binding domain (e.g., a sequence-specific DNA binding domain from a polynucleotide-guided endonuclease, a zinc finger nuclease, a transcription activator-like effector nuclease (TALEN), an Argonaute proteiw and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CR1SPR-Cas endonuclease (e.g. CR1SPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be codon optimized for expression in a plant. In some embodiments, the codon optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been codon optimized.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length, for example, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 2 to about 40, about 2 to about 50, about 2 to about 60, about 4 to about 40, about 4 to about 50, about 4 to about 60, about 5 to about 40, about 5 to about 50, about 5 to about 60, about 9 to about 40, about 9 to about 50, about 9 to about 60, about 10 to about 40, about 10 to about 50, about 10 to about 60, or about 2, 3, 4, 5, 6, 7, 8, 9, 10, 11, 12, 13, 14, 15, 16, 17, 18, 19, 20, 21, 22, 23, 24, 25 amino acids to about 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length (e.g., about 105, 110, 115, 120, 130, 140 150 or more amino acids in length). In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g., extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2 USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA2-6 promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18.2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific DNA binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific DNA binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific DNA binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific DNA binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific DNA binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific DNA binding protein (e.g., polynucleotide-guided endonuclease, a CR1SPR-Cas endonuclease (e.g. CR1SPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific DNA binding protein, the reverse transcriptase and/or the deaminase are expressed and the sequence-specific DNA binding protein binds to the target nucleic acid, and the reverse transcriptase and/or deaminase may be fused to either the sequence-specific DNA binding protein or recruited to the sequence-specific DNA binding protein (via, for example, a peptide tag fused to the sequence-specific DNA binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

Morphological studies of developing Rubus prickles suggest that they are modified glandular trichomes. Additionally, the correlation between the presence of glandular trichomes and the presence of prickles in blackberry, for example, suggest that glandular trichomes are indeed involved in blackberry prickle development. The trichome developmental pathway is well studied in Arabidopsis thaliana with many transcription factors and regulators identified. Arabidopsis does not have glandular trichomes and this pathway discovery has focused on the development of non-glandular trichomes. The regulatory control of glandular trichome development may be different for non-glandular trichomes, although initiation mechanisms may be shared. In addition to Arabidopsis, several other genes have been identified as causing the absence of trichomes in other plant species.

As described herein, editing technology is used to target MIXTA transcription factor genes to generate thornless or prickleless Rubus plants. In some aspects, the mutation will be a dominant negative mutation, which may be advantageous when the plant is polyploidy, such as blackberry. In some embodiments, mutations may be generated by truncating the MIXTA transcription factor polypeptides. Other types of mutations useful for production of thornless/prickleless Rubus plants include substitution, deletion and insertion.

In some embodiments, a Rubus plant or plant part thereof is provided, the Rubus plant or plant part comprising at least one non-natural mutation (e.g., 1, 2, 3, 4, 5, or more mutations) in at least one copy of an endogenous gene encoding a MIXTA transcription factor. In some embodiments, at least one non-natural mutation in the Rubus plant or plant part may be a null allele. In some embodiments, at least one non-natural mutation in the Rubus plant or plant part may be a dominant negative mutation. In some embodiments, a Rubus plant or plant part comprises at least two (at least four, at least six, at least eight, at least ten, at least twelve, e.g., 2, 4, 6, 8, 10, 12, 14 or more) copies of the endogenous gene encoding a MIXTA transcription factor and each copy comprises at least one non-natural mutation, optionally wherein the mutation is a null allele or a dominant negative mutation. In some embodiments, the at least one or at least two non-natural mutation is a base substitution, a deletion and/or an insertion as described herein. A Rubus plant or part thereof includes, but is not limited to, a blackberry, black raspberry or raspberry. In some embodiments, the Rubus plant is a blackberry plant that comprises four copies of the endogenous MIXTA transcription factor gene, and each copy is mutated/edited as described herein. In some embodiments, the Rubus plant is a blackberry plant that comprises six copies of the endogenous MIXTA transcription factor gene, and each copy is mutated/edited as described herein. In some embodiments, the Rubus plant is a blackberry plant that comprises eight copies of the endogenous MIXTA transcription factor gene, and each copy is mutated/edited as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele.

In some embodiments, a Rubus plant cell is provided, comprising an editing system comprising: (a) a CRISPR-associated effector protein; and (b) a guide nucleic acid having a spacer sequence with complementarity to an endogenous target gene encoding a wild type MIXTA transcription factor. The wild type MIXTA transcription factor may be any MIXTA transcription factor involved in the development of thorns/prickles. In some embodiments, the wild type MIXTA transcription factor gene to which the spacer sequence of the guide nucleic acid shares complementarity may encode a polypeptide comprising at least 90% sequence identity (e.g., about 90, 91, 92, 93, 94, 95, 96, 97, 98, 99, 99.5, 100% sequence identity) to any one of the amino acid sequences of SEQ ID NOs:57-62, 71-72, 94, 96, 98, or 100, a polypeptide comprising at least 94% sequence identity (e.g., about 94, 95, 96, 97, 98, 99, 99.5, 100% sequence identity) to the amino acid sequence of SEQ ID NO:57, or a polypeptide that comprises the amino acid sequence of any one of SEQ ID NOs:73-76 or 91-92, or the MIXTA transcription factor may comprise the nucleotide sequence of any one of SEQ ID NOs:63-70, 93, 95, 97, or 99, a nucleotide sequence that comprises any one of SEQ ID NOs:77 or 84-89, or a nucleotide sequence having at least 95% sequence identity (e.g., about 95, 96, 97, 98, 99, 99.5, 100% sequence identity) to the nucleotide sequence of SEQ ID NO:78 or SEQ ID NO:83. In some embodiments, a spacer sequence of a guide nucleic acid of an editing system of this invention may comprise a nucleotide sequence of any one of SEQ ID NOs:53-56 or 109-117. In some embodiments, the Rubus plant cell is from, for example, a blackberry plant, a black raspberry plant or a raspberry plant. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant is a blackberry plant that comprises four copies of the endogenous MIXTA transcription factor gene, and each copy is mutated/edited as described herein. In some embodiments, the Rubus plant is a blackberry plant that comprises six copies of the endogenous MIXTA transcription factor gene, and each copy is mutated/edited as described herein. In some embodiments, the Rubus plant is a blackberry plant that comprises eight copies of the endogenous MIXTA transcription factor gene, and each copy is mutated/edited as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene.

In some embodiments, the invention provides a Rubus plant cell comprising at least one non-naturally occurring mutation within a MIXTA transcription factor gene that results in a null allele or knockout of the MIXTA transcription factor gene, or results in a dominant negative mutation of the MIXTA transcription factor gene, wherein the mutation is a substitution, insertion or a deletion that is introduced using an editing system that comprises a nucleic acid binding domain that binds to a target site in the MIXTA transcription factor gene, wherein the MIXTA transcription factor gene comprises a nucleotide sequence of any one of SEQ ID NOs: 63-70, 77, 84-89, 93, 95, 97, or 99, or a nucleotide sequence having at least 95% identity (e.g., about 95, 96, 97, 98, 99, 99.5, 100% sequence identity) to the nucleotide sequence of SEQ ID NO:78 or SEQ ID NO:83, or encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57. In some embodiments, a Rubus plant cell comprises at least two (or at least four, at least six, at least eight, at least ten, at least twelve, e.g., 2, 4, 6, 8, 10, 12, or 14 or more) copies of the endogenous gene encoding a MIXTA transcription factor and each copy comprises at least one non-natural mutation, optionally wherein the mutation is a null allele or a dominant negative mutation. The mutation in the MIXTA transcription factor gene of the Rubus plant cell may be any mutation of a MIXTA transcription factor or the gene encoding the MIXTA transcription factor as described herein, including a deletion, substitution or insertion. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the non-natural mutation is a null allele and results in a truncation of the MIXTA transcription factor, for example, a C-terminal truncation. In some embodiments, the mutation is a dominant negative mutation and results in a truncation of the MIXTA transcription factor, for example, a C-terminal truncation. In some embodiments, the nucleic acid binding domain of an editing system useful with this invention may be from a polynucleotide-guided endonuclease, a CR1SPR-Cas endonuclease CR1SPR-Cas effector protein), a zinc finger nuclease, a transcription activator-like effector nuclease (TALEN) and/or an Argonaute protein. In some embodiments, a plant cell edited as described herein may be regenerated into a plant, thereby providing a plant with a mutation in the MIXTA transcription factor that is involved in thorn/prickle development and having reduced thorns/prickles as compared to a plant not comprising the mutation in the MIXTA transcription factor. In some embodiments, the Rubus plant cell is from, for example, a blackberry plant, a black raspberry plant or a raspberry plant. In some embodiments, the Rubus plant cell is from a blackberry plant, which cell comprises four copies of the endogenous MIXTA transcription factor gene, and each copy in the cell is edited and comprises a mutation/edit as described herein. In some embodiments, the Rubus plant cell is from a blackberry plant, which cell comprises six copies of the endogenous MIXTA transcription factor gene, and each copy in the cell is edited and comprises a mutation/edit as described herein. In some embodiments, the Rubus plant cell is from a blackberry plant, which cell comprises eight copies of the endogenous MIXTA transcription factor gene, and each copy in the cell is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene.

In some embodiments, a method of producing/breeding a transgene-free edited Rubus plant, comprising: crossing the Rubus plant of the present invention (e.g., a Rubus plant comprising a mutation in a MIXTA transcription factor and having reduced thorns and/or prickles) with a transgene free Rubus plant, thereby introducing (e.g., segregating, selfing, etc.) the at least one non-natural mutation into the Rubus plant that is transgene-free; and selecting a progeny Rubus plant that comprises the at least one non-natural mutation and is transgene-free, thereby producing a transgene free edited Rubus plant. In some embodiments, a Rubus plant is, for example, a blackberry plant, a black raspberry plant or a raspberry plant.

Also provided herein is a method of providing a plurality of Rubus plants (e.g., blackberry, black raspberry, raspberry) having reduced thorns and/or reduced prickleness, the method comprising planting two or more Rubus plants of the invention (e.g., 2, 3, 4, 5, 6, 7, 8, 9, 10, 20, 30, 40, 50, 100, 150, or 200 or more Rubus plants comprising a mutation in a MIXTA transcription factor as described herein and having reduced thorns and/or prickles) in a growing area (e.g., an agricultural field, greenhouse, and the like), thereby providing a plurality of Rubus plants having reduced thorns and/or reduced prickles as compared to a plurality of control Rubus plants not comprising the mutation.

In some embodiments, a method for editing a specific site in the genome of a Rubus plant cell (e.g., a Rubus plant cell from, for example, a black raspberry plant, a blackberry plant, or a red raspberry plant) is provided, the method comprising cleaving, in a site specific manner, a target site within an endogenous MIXTA transcription factor gene in the Rubus plant cell, the endogenous MIXTA transcription factor gene comprising the nucleotide sequence of any one of SEQ ID NOs: 63-70, 77, 84-89, 93, 95, 97, or 99 or a nucleotide sequence having at least 95% identity to the nucleotide sequence of or SEQ ID NO:83, or encoding a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57, thereby generating an edit in the endogenous MIXTA transcription factor gene of the Rubus plant cell and producing a plant cell comprising the edit in the endogenous MIXTA transcription factor gene. In some embodiments, the Rubus plant cell comprises at least two (or at least four, at least six, at least eight, at least ten, at least twelve, e.g., 2, 4, 6, 8, 10, 12, or 14 or more) copies of the endogenous gene encoding a MIXTA transcription factor and each copy comprises at least one non-natural mutation, optionally wherein the mutation is a null allele or a dominant negative mutation. In some embodiments, the edit results in a non-naturally occurring mutation including, but not limited to, a deletion, substitution, or insertion as described herein, optionally wherein the edit results in a null allele or in a dominant negative mutation. In some embodiments, the edit is a base deletion or a substitution that results in a truncation of the MIXTA transcription factor polypeptide as described herein. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the method may further comprise regenerating a Rubus plant from the Rubus plant cell comprising the edit in the endogenous MIXTA transcription factor gene (or in the at least two copies (e.g., in 2, 4, 6, 8, 10, 12, or 14 or more) of the endogenous MIXTA transcription factor gene), thereby producing a Rubus plant comprising the edit in the endogenous MIXTA transcription factor gene and having reduced thorns and/or prickles compared to a control Rubus plant that does not comprise the edit. In some embodiments, the Rubus plant cell is from a blackberry plant, which cell comprises four copies of the endogenous MIXTA transcription factor gene, and each copy in the cell is edited and comprises a mutation/edit as described herein. In some embodiments, the Rubus plant cell is from a blackberry plant, which cell comprises six copies of the endogenous MIXTA transcription factor gene, and each copy in the cell is edited and comprises a mutation/edit as described herein. In some embodiments, the Rubus plant cell is from a blackberry plant, which cell comprises eight copies of the endogenous MIXTA transcription factor gene, and each copy in the cell is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies of the MIXTA gene to produce a plant comprising an edit in each copy of the MIXTA gene.

In some embodiments, a method for making a Rubus plant comprising a mutation in an endogenous gene is provided, the method comprising: (a) contacting a population of Rubus plant cells comprising at least one wild type endogenous MIXTA transcription factor gene with a nuclease linked to a DNA binding domain (e.g., an editing system) [and a guide nucleic acid comprising a spacer having complementarity to the target site] that binds to a target site in the at least one wild type endogenous MIXTA transcription factor gene, wherein the at least one wild type endogenous MIXTA transcription factor gene: (i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83; (b) selecting a Rubus plant cell from said population that comprises a mutation (e.g., at least one mutation, e.g., 1, 2, 3, 4, 5, or more mutations) in the endogenous MIXTA transcription factor gene, wherein the mutation results in a null allele or a dominant negative mutation of the endogenous MIXTA transcription factor gene; and (c) growing the selected Rubus plant cell into a Rubus plant comprising the mutation of the endogenous MIXTA transcription factor gene. In some embodiments, the mutation is a base deletion or a substitution that results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant comprises 2 or more copies (or in at least two, e.g., 2, 4, 6, 8, 10, 12, or 14 or more copies) of the endogenous MIXTA transcription factor gene and each copy comprises a mutation/edit as described herein. In some embodiments, the Rubus plant cell is from, for example, a blackberry plant, a black raspberry plant or a raspberry plant and the plant that is produced is a blackberry plant, a black raspberry plant or a raspberry plant, respectively. In some embodiments, the Rubus plant cell is from a blackberry plant, which cell comprises four copies of the endogenous MIXTA transcription factor gene, and each copy in the cell is edited and comprises a mutation/edit as described herein. In some embodiments, the Rubus plant cell is from a blackberry plant, which cell comprises six copies of the endogenous MIXTA transcription factor gene, and each copy in the cell is edited and comprises a mutation/edit as described herein. In some embodiments, the Rubus plant cell is from a blackberry plant, which cell comprises eight copies of the endogenous MIXTA transcription factor gene, and each copy in the cell is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in the one or more copies to produce a plant comprising an edit in each copy of the MIXTA gene.

In some embodiments, a method is provided for reducing thorns and/or prickles in a Rubus plant or part thereof, the method comprising (a) contacting a plant cell comprising a wild type endogenous MIXTA transcription factor gene with a nuclease targeted to the wild type endogenous MIXTA transcription factor gene [and a guide nucleic acid comprising a spacer having complementarity to the target site], wherein the nuclease is linked to a DNA binding domain that binds to a target site in the endogenous MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a nucleotide sequence having at least 95% identity to the nucleotide sequence of SEQ ID NO:78 or SEQ ID NO:83, thereby producing a Rubus plant or part thereof having reduced thorns and/or prickles. In some embodiments, the mutation is a base deletion or a substitution that results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant cell that is contacted may comprise at least two copies of the endogenous MIXTA transcription factor gene (e.g., 2, 4, 6, 8, 10, 12, or 14, or more copies) and each copy comprises at least one mutation as described herein, thereby producing a Rubus plant or part thereof having reduced thorns and/or prickles. In some embodiments, the Rubus plant cell is from, for example, a blackberry plant, a black raspberry plant or a raspberry plant and the plant that is produced is a blackberry plant, a black raspberry plant or a raspberry plant, respectively. In some embodiments, the Rubus plant cell is from a blackberry plant, which cell comprises four copies of the endogenous MIXTA transcription factor gene, and each copy in the cell is edited and comprises a mutation/edit as described herein. In some embodiments, the Rubus plant cell is from a blackberry plant, which cell comprises six copies of the endogenous MIXTA transcription factor gene, and each copy in the cell is edited and comprises a mutation/edit as described herein. In some embodiments, the Rubus plant cell is from a blackberry plant, which cell comprises eight copies of the endogenous MIXTA transcription factor gene, and each copy in the cell is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies of the MIXTA gene to produce a plant comprising an edit in each copy of the MIXTA gene.

In some embodiments, a method for reducing thorns and prickles in a Rubus plant or part thereof (e.g., a blackberry plant, a black raspberry plant or a raspberry plant) is provided, comprising (a) contacting a Rubus plant cell comprising a wild type endogenous MIXTA transcription factor gene with a nuclease targeting the wild type endogenous MIXTA transcription factor gene [and a guide nucleic acid comprising a spacer having complementarity to the target site], wherein the nuclease is linked to a DNA binding domain that binds to a target site in the wild type endogenous MIXTA transcription factor gene, wherein the wild type endogenous MIXTA transcription factor gene: (i) encodes a polypeptide comprising the sequence of any one of the amino acid sequences of 57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83; (b) growing the plant cell into a plant, thereby reducing thorns and prickles in the Rubus plant or part thereof as compared to a Rubus plant or part thereof that has not been contacted with the nuclease. In some embodiments, contacting the Rubus plant cell comprising a wild type endogenous MIXTA transcription factor gene with a nuclease targeting the wild type endogenous MIXTA transcription factor gene produces a mutation that is a base deletion or a substitution, which results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant cell that is contacted may comprise at least two copies of an endogenous MIXTA transcription factor gene (e.g., 2, 4, 6, 8, 10, 12, or 14, or more copies) and each copy comprises at least one mutation as described herein, thereby reducing thorns and prickles in the Rubus plant or part thereof. In some embodiments, the Rubus plant cell is from a blackberry plant, which cell comprises four copies of the endogenous MIXTA transcription factor gene, and each copy in the cell is edited and comprises a mutation/edit as described herein. In some embodiments, the Rubus plant cell is from a blackberry plant, which cell comprises six copies of the endogenous MIXTA transcription factor gene, and each copy in the cell is edited and comprises a mutation/edit as described herein. In some embodiments, the Rubus plant cell is from a blackberry plant, which cell comprises eight copies of the endogenous MIXTA transcription factor gene, and each copy in the cell is edited and comprises a mutation/edit as described herein. When multiple copies of the MIXTA gene are present in a genome the edit may be introduced into each copy of a MIXTA gene by editing as described herein, orb by editing one or more copies of the MIXTA gene in a plant and then selfing the plant comprising the edit in one or more copies to produce a plant comprising an edit in each copy of the MIXTA gene.

In some embodiments, a method for producing a Rubus plant or part thereof comprising at least one cell having a mutated endogenous MIXTA transcription factor gene is provided, the method comprising contacting a target site in an endogenous MIXTA transcription factor gene in the Rubus plant or plant part with a nuclease comprising a cleavage domain and a DNA-binding domain [and a guide nucleic acid comprising a spacer having complementarity to the target site], wherein the DNA binding domain binds to a target site in the endogenous MIXTA transcription factor gene, wherein the endogenous MIXTA transcription factor gene (i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a nucleotide sequence having at least 95% identity to the nucleotide sequence of SEQ ID NO:78 or SEQ ID NO:83, thereby producing the Rubus plant or part thereof comprising at least one cell having a mutation in the endogenous MIXTA transcription factor gene. In some embodiments, contacting a target site in an endogenous MIXTA transcription factor gene with the nuclease produces a mutation that is a base deletion or a substitution, which results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant or plant part comprises at least two copies of an endogenous MIXTA transcription factor gene (e.g., 2, 4, 6, 8, 10, 12, or 14, or more copies) and each copy comprises a target site that can be contacted with the nuclease to produce a mutation as described herein in each copy, thereby producing the Rubus plant or part thereof comprising at least one cell having a mutation in at least two copies of the endogenous MIXTA transcription factor gene. In some embodiments, the Rubus plant is, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part is edited and comprises a mutation/edit as described herein. In some embodiments, the Rubus plant is a blackberry plant, which comprises six copies of the endogenous MIXTA transcription factor gene, and each copy in the blackberry plant or plant part is edited and comprises a mutation/edit as described herein. In some embodiments, the Rubus plant is a blackberry plant, which comprises eight copies of the endogenous MIXTA transcription factor gene, and each copy in the blackberry plant or plant part is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies of the MIXTA gene to produce a plant comprising an edit in each copy of the MIXTA gene.

Also provided herein is a method for producing a Rubus plant or part thereof comprising a mutated endogenous MIXTA transcription factor and reduced thorns and/or prickles, the method comprising contacting a target site in an endogenous MIXTA transcription factor gene in the Rubus plant or plant part with a nuclease comprising a cleavage domain and a DNA-binding domain [and a guide nucleic acid comprising a spacer having complementarity to the target site], wherein the DNA binding domain binds to a target site in the endogenous MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs:63-70, 77, 84-89, 93, 95, 97, or 99 or a nucleotide sequence having at least 95% identity to the nucleotide sequence of SEQ ID NO:78 or SEQ ID NO:83, thereby producing the Rubus plant or part thereof comprising a mutated endogenous MIXTA transcription factor and reduced thorns and/or prickles as compared to a Rubus plant or part thereof that has not been contacted with the nuclease comprising a cleavage domain and a DNA-binding domain. In some embodiments, contacting a target site in an endogenous MIXTA transcription factor gene with the nuclease produces a mutation that is a base deletion or a substitution, which results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant or plant part comprises at least two copies of an endogenous MIXTA transcription factor gene (e.g., 2, 4, 6, 8, 10, 12, or 14, or more copies) and each copy comprises a target site that can be contacted with the nuclease to produce a mutation as described herein in each copy, thereby producing the Rubus plant or part thereof comprising a mutation in at least two copies of the endogenous MIXTA transcription factor gene and reduced thorns and/or prickles. In some embodiments, the Rubus plant is, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part is edited and comprises a mutation/edit as described herein. In some embodiments, the Rubus plant is a blackberry plant, which comprises six copies of the endogenous MIXTA transcription factor gene, and each copy in the blackberry plant or plant part is edited and comprises a mutation/edit as described herein. In some embodiments, the Rubus plant is a blackberry plant, which comprises eight copies of the endogenous MIXTA transcription factor gene, and each copy in the blackberry plant or plant part is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies of the MIXTA gene to produce a plant comprising an edit in each copy of the MIXTA gene.

In some embodiments, a method is provided for producing a Rubus plant or part thereof comprising at least one cell having a mutated endogenous MIXTA transcription factor gene, the method comprising contacting a target site in an endogenous MIXTA transcription factor gene in the Rubus plant or plant part with a nuclease comprising a cleavage domain and a DNA-binding domain [and a guide nucleic acid comprising a spacer having complementarity to the target site], wherein the DNA binding domain binds to the target site in the MIXTA transcription factor gene, wherein the endogenous MIXTA transcription factor gene: (i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83, thereby producing a Rubus plant or part thereof comprising at least one cell having a mutated endogenous MIXTA transcription factor gene and having reduced thorns and/or prickles as compared to a Rubus plant or part thereof that has not been contacted with the nuclease comprising a cleavage domain and a DNA-binding domain. In some embodiments, contacting a target site in an endogenous MIXTA transcription factor gene with the nuclease produces a mutation that is a base deletion or a substitution, which results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele. In some embodiments, the Rubus plant or plant part comprises at least two copies of an endogenous MIXTA transcription factor gene (e.g., 2, 4, 6, 8, 10, 12, or 14, or more copies) and each copy comprises a target site that can be contacted with the nuclease to produce a mutation as described herein in each copy, thereby producing a Rubus plant or part thereof comprising at least one cell having a mutation in at least two copies of the endogenous MIXTA transcription factor gene and having reduced thorns and/or prickles as compared to a Rubus plant or part thereof that has not been contacted with the nuclease comprising a cleavage domain and a DNA-binding domain. In some embodiments, the Rubus plant is, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part thereof is edited and comprises a mutation/edit as described herein. In some embodiments, the Rubus plant is a blackberry plant, which comprises six copies of the endogenous MIXTA transcription factor gene, and each copy in the blackberry plant or plant part thereof is edited and comprises a mutation/edit as described herein. In some embodiments, the Rubus plant is a blackberry plant, which comprises eight copies of the endogenous MIXTA transcription factor gene, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies of the MIXTA gene to produce a plant comprising an edit in each copy of the MIXTA gene.

A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any DNA binding domain useful with the invention may be any DNA binding domain that can be utilized to edit/modify a target nucleic acid. Such DNA binding domains include, but are not limited to, a zinc finger, transcription activator-like DNA binding domain (TAL), an argonaute and/or a CRISPR-Cas effector DNA binding domain.

In some embodiments, a method of editing an endogenous MIXTA transcription factor gene in a plant or plant part is provided, the method comprising contacting a target site in the MIXTA transcription factor gene in the plant or plant part with a cytosine base editing system comprising a cytosine deaminase and a nucleic acid binding domain that binds to a target site in the MIXTA transcription factor, the MIXTA transcription factor gene comprising the nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83, or encoding a polypeptide comprising the sequence of any one of the amino acid sequences of SEQ ID NOs: SEQ ID NOs:57-62, 71-76, 90-92, 94, 96, 98, or 100 or a sequence having at least at least 94% identity to any one of the amino acid sequences of SEQ ID NO:57, thereby editing the endogenous MIXTA transcription factor gene in the plant or part thereof and producing a plant or part thereof comprising at least one cell having a mutation in the endogenous MIXTA transcription factor gene. In some embodiments, the endogenous MIXTA transcription factor gene is present in the plant or plant part in multiple copies (e.g., in 2, 4, 6, 8, 10, 12, or 14, or more copies) and each copy comprises a mutation as described herein. In some embodiments, the plant is a Rubus plant, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part thereof is edited and comprises a mutation/edit as described herein. In some embodiments, the Rubus plant is a blackberry plant, which comprises six copies of the endogenous MIXTA transcription factor gene, and each copy in the blackberry plant or plant part thereof is edited and comprises a mutation/edit as described herein. In some embodiments, the Rubus plant is a blackberry plant, which comprises eight copies of the endogenous MIXTA transcription factor gene, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies in a plant as described herein and then selfing the plant comprising the edit in one or more copies of the MIXTA gene to produce a plant comprising an edit in each copy of the MIXTA gene.

In some embodiments, a method of editing an endogenous MIXTA transcription factor gene in a plant or plant part is provided, the method comprising contacting a target site in the MIXTA transcription factor gene in the plant or plant part with a adenosine base editing system comprising an adenosine deaminase and a nucleic acid binding domain that binds to a target site in the MIXTA transcription factor, the MIXTA transcription factor gene comprising the nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83, or encoding a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57, thereby editing the endogenous MIXTA transcription factor gene in the plant or part thereof and producing a plant or part thereof comprising at least one cell having a mutation in the endogenous MIXTA transcription factor gene. In some embodiments, the endogenous MIXTA transcription factor gene is present in the plant or plant part in multiple copies (e.g., in 2, 4, 6, 8, 10, 12, or 14, or more copies) and each copy comprises a mutation as described herein. In some embodiments, the plant is a Rubus plant, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part thereof is edited and comprises a mutation/edit as described herein. In some embodiments, the Rubus plant is a blackberry plant, which comprises six copies of the endogenous MIXTA transcription factor gene, and each copy in the blackberry plant or plant part thereof is edited and comprises a mutation/edit as described herein. In some embodiments, the Rubus plant is a blackberry plant, which comprises eight copies of the endogenous MIXTA transcription factor gene, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies of a MIXTA gene in a plant as described herein and then selfing the plant comprising the edit in one or more copies of the MIXTA gene to produce a plant comprising an edit in each copy of the MIXTA gene.

In some embodiments, contacting a target site in an endogenous MIXTA transcription factor gene with an adenosine or cytosine base editing system produces a mutation that is a base deletion or a substitution, which results in a truncation of the MIXTA transcription factor polypeptide as described herein. In some embodiments, the mutation results in a null allele or is a dominant negative mutation of the endogenous MIXTA transcription factor gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele.

In some embodiments, a method of detecting a mutant MIXTA (a mutation in an endogenous MIXTA transcription factor gene) is provided, the method comprising detecting in the genome of a plant a deletion in a nucleic acid encoding the amino acid sequence of any one of SEQ ID NOs:57-62, 71-72, 94, 96, 98, or 100, wherein the amino acid sequence of any one of SEQ ID NOs: 57-62, 71-72, 94, 96, 98, or 100 comprises a truncation of consecutive amino acid residues from amino acid residue 25, 26, 27, 28, 29, 30, 31, 32, 33, 34, 35, 36, 37, 38, 39, 40, 45, 50, 55, 60, 65, 70, 75, 80, 85, 90, 95, 100 to amino acid residue 401, 412, 413, 415, or 417 of any one of SEQ ID NOs:57-62, 71-72, 94, 96, 98, or 100.

In some embodiments, a method of detecting a mutant MIXTA (a mutation in an endogenous MIXTA transcription factor gene) is provided, the method comprising detecting in the genome of a plant a deletion in the nucleotide sequence of any one of SEQ ID NOs:63-70, 93, 95, 97, or 99, wherein the nucleotide sequence of any one of SEQ ID NOs:63-70, 93, 95, 97, or 99 comprises a deletion of consecutive nucleotides from nucleotide 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 100, 110, 120, 130, 140, 150, 200, 250, 300 to nucleotide 1200, 1201, 1202, 1203, 1204, 1205, 1206, 1207, 208, 1209, 1210, 1215, 1220, 1225, 1230, 1235, 1240, 1241, 1242, 1242, 1243, 1244, 1245, 1246, 1247, 1248, 1249, 1250, 1251, 1252, 1253, or 1254 of any one of SEQ ID NOs:63-70, 93, 95, 97, or 99.

In some embodiments, a method of detecting a mutant MIXTA (a mutation in an endogenous MIXTA transcription factor gene) is provided, the method comprising detecting in the genome of a plant a substitution in a nucleic acid encoding the amino acid sequence of any one of SEQ ID NOs:57-62, 71-72, 94, 96, 98, or 100, wherein the substitution in the nucleic acid results in an premature stop codon and thus, an amino acid sequence comprising a truncation of consecutive amino acid residues from amino acid residue 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100 to amino acid residue 401, 412, 413, 415, or 417 of any one of SEQ ID NOs:57-62, 71-72, 94, 96, 98, or 100.

In some embodiments, a method of detecting a mutant MIXTA (a mutation in an endogenous MIXTA transcription factor gene) is provided, the method comprising detecting in the genome of a plant a base substitution of one or more of nucleotides in the nucleotide sequence of any one of SEQ ID NOs:63-70, 93, 95, 97, or 99.

In some embodiments, the present invention provides a method of detecting a mutation in an endogenous MIXTA gene, comprising detecting in the genome of a plant a mutated MIXTA gene produced as described herein (see, e.g., SEQ ID NOs:101-107).

In some embodiments, the present invention provides a method of producing a plant comprising a mutation in an endogenous MIXTA transcription factor gene and at least one polynucleotide of interest, the method comprising crossing a plant of the invention comprising at least one mutation in an endogenous MIXTA transcription factor gene (a first plant) with a second plant that comprises the at least one polynucleotide of interest to produce progeny plants; and selecting progeny plants comprising at least one mutation in the MIXTA transcription factor gene and the at least one polynucleotide of interest, thereby producing the plant comprising a mutation in an endogenous MIXTA transcription factor gene and at least one polynucleotide of interest.

The present invention further provides a method of producing a plant comprising a mutation in an endogenous MIXTA transcription factor gene and at least one polynucleotide of interest, the method comprising introducing at least one polynucleotide of interest into a plant of the present invention comprising at least one mutation in a MIXTA transcription factor gene, thereby producing a plant comprising at least one mutation in a MIXTA transcription factor gene and at least one polynucleotide of interest.

In some embodiments, the present invention provides a method of producing a plant comprising a mutation in an endogenous MIXTA transcription factor gene and at least one polynucleotide of interest, the method comprising introducing at least one polynucleotide of interest into a plant of the invention comprising at least one mutation in an endogenous MIXTA transcription factor gene, thereby producing a plant comprising at least one mutation in a MIXTA transcription factor gene and at least one polynucleotide of interest.

A polynucleotide of interest may be any polynucleotide that can confer a desirable phenotype or otherwise modify the phenotype or genotype of a plant. In some embodiments, a polynucleotide of interest may be polynucleotide that confers herbicide tolerance, insect resistance, disease resistance, increased yield, increased nutrient use efficiency or abiotic stress resistance.

A MIXTA transcription factor useful with this invention includes any MIXTA transcription factor capable of regulating thorn or prickle production in a Rubus plant or part thereof. In some embodiments, the MIXTA transcription factor comprises an amino acid sequence having at least 90% identity (e.g., about 90, 91, 92, 93, 94, 95, 96, 97, 98, 99, 99.5, 100% sequence identity) to any one of SEQ ID NOs:57-62, 71, 72, 94, 96, 98, or 100 or comprises an amino acid sequence comprising any one of SEQ ID NOs:73-76, 90, 91, or 92. In some embodiments, an endogenous gene encoding a MIXTA transcription factor comprises a nucleotide sequence any one of SEQ ID NOs:63-70, 77, 84-89, 93, 95, 97, or 99, or a nucleotide sequence that comprises a sequence having at least 95% identity (e.g., about 95, 96, 97, 98, 99, 99.5, 100% sequence identity) to the nucleotide sequence of SEQ ID NOs:78 or SEQ ID NO:83. In some embodiments, the gene encoding the MIXTA transcription factor may be present in at least two copies (e.g., 2, 4, 6, 8, 10, 12, or 14, or more copies), wherein each copy comprises a mutation as described herein. In some embodiments, the Rubus plant is, for example, a blackberry plant, a black raspberry plant or a raspberry plant. In some embodiments, the Rubus plant is a blackberry plant and the endogenous MIXTA transcription factor gene is present in four copies, and each copy in the blackberry plant or plant part thereof is edited and comprises a mutation/edit as described herein. In some embodiments, the Rubus plant is a blackberry plant and the endogenous MIXTA transcription factor gene is present in six copies, and each copy in the blackberry plant or plant part thereof is edited and comprises a mutation/edit as described herein. In some embodiments, the Rubus plant is a blackberry plant and the endogenous MIXTA transcription factor gene is present in eight copies, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies of a MIXTA gene in a plant as described herein and then selfing the plant comprising the edit in one or more copies to produce a plant comprising an edit in each copy of the MIXTA gene.

Any mutation in a MIXTA transcription factor gene that produces a non-functional MIXTA transcription factor may be used to produce Rubus plants (e.g., blackberry, black raspberry, or raspberry) or parts thereof of this invention having a reduced number of or no thorns or prickles. In some embodiments, the mutation in the MIXTA transcription factor gene may produce a MIXTA transcription factor that is reduced in functionality (e.g., attenuated ability to function in its role in the development of thorns and prickles) may be used to produce Rubus plants or parts thereof of this invention having a reduced number of or no thorns or prickles.

In some embodiments, the at least one non-natural mutation in an endogenous MIXTA transcription factor gene is a null allele (e.g., produces a non-functional protein or no protein). In some embodiments, the at least one non-natural mutation in an endogenous MIXTA transcription factor gene is a dominant negative mutation (e.g., produces a protein having aberrant function that interferes with the function wild type gene product). In some embodiments, the at least one non-natural mutation in an endogenous MIXTA transcription factor gene in a Rubus plant may be a base substitution, a deletion and/or an insertion. In some embodiments, the at least one non-natural mutation in an endogenous MIXTA transcription factor gene in a Rubus plant may be a base substitution, a deletion and/or an insertion that results in a null allele and a Rubus plant having reduced thorns and/or prickles. In some embodiments, the at least one non-natural mutation in an endogenous MIXTA transcription factor gene in a Rubus plant may be a substitution, a deletion and/or an insertion that results in a dominant negative mutation and a Rubus plant having reduced thorns and/or prickles. For example, the mutation may be a substitution, a deletion and/or an insertion of one or more amino acid residues (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids of the transcription factor) or the mutation may be a substitution, a deletion and/or an insertion of at least 2 nucleotides (e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350, 400, 450, 500, 550, 600, 650, 700, 750, 800, 850, 900, 950, 1000, 1100, 1200, 1210, 1220, 1230, 1240 or 1250 or more nucleotides, or any range or value therein) (e.g., a base substitution, deletion and/or insertion) from the gene encoding the transcription factor. In some embodiments, the at least one non-natural mutation may be a base substitution to an A, a T, a G, or a C. As described herein, a Rubus plant or plant cell (e.g., blackberry, black raspberry, or raspberry) may comprise multiple copies (e.g., in 2, 4, 6, 8, 10, 12, or 14, or more copies) of the endogenous MIXTA transcription factor gene and each copy in the plant or plant cell may comprise at least one non-natural mutation as described herein. That is, all copies of the endogenous MIXTA transcription factor gene present in the Rubus plant are mutated as described herein, whether there are 2, 4, 6, 8, 10, 12, or 14, or more copies present in the Rubus plant. In some embodiments, the Rubus plant is a blackberry plant and the blackberry plant comprises 2, 4, 6 or 8 or more copies of the endogenous MIXTA transcription factor gene in its genome, wherein each copy comprises a mutation as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene. In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele.

In some embodiments, a mutation in a MIXTA transcription factor produced by methods of this invention may be a deletion. In some embodiments, a mutation in a MIXTA transcription factor produced by methods of this invention may be the result of a base pair substitution in the encoding gene. In some embodiments, a deletion and/or a substitution may result in a truncation of the MIXTA transcription factor polypeptide. In some embodiments, the mutation may be an N-terminal truncation. In some embodiments, the mutation is a C-terminal truncation. When the mutation of the MIXTA transcription factor is a C-terminal truncation, the C-terminal truncation may comprise a truncation of at least 1 amino acid residue (e.g., about 1, about 5, about 10, about 15, or about 20 amino acid residue to about 300, about 310, about 320, about 330, about 340, or about 350 consecutive amino acid residues or more) (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 230, 330, 340, 350, 360, 370, 380, or 400 consecutive amino acid residues, or more, or any range or value therein) from the C-terminus of the MIXTA transcription factor (e.g., SEQ ID NOs:57-62, 71, 72, 94, 96, 98, or 100). In some embodiments, when the mutation of the MIXTA transcription factor is a C-terminal truncation, the polynucleotide encoding the truncated MIXTA transcription factor polypeptide may comprise a deletion or a substitution of at least 2 consecutive base pairs (e.g., about 2, 3, 4, 5, 6, 7, 8, 9, 10, 11, 12, 13, 14, 15, 16, 17, 18, 19, 20, 21, 22, 23, 24, 25, 30, 40, 50, 100 consecutive base pairs to about 150, 200, 300, 400, 500, 600, 700, 800, 900, or 950 consecutive base pairs; e.g., about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350, 400, 450, 500, 550, 600, 650, 700, 750, 800, 850, 900, 950, 1000, 1100, 1200, 1210, 1220, 1230, 1240 or 1250 or more consecutive base pairs, or any range or value therein; optionally a substation of 2 base pairs and/or a deletion of about 5 to about 25 base pairs) from an endogenous gene encoding the MIXTA transcription factor (e.g., SEQ ID NOs:63-70, 93, 95, 97, or 99). In some embodiments, a deletion that produces a truncated protein may be about 5 to about 25 nucleotides near the 5′ end of the MIXTA transcription factor gene resulting in a premature stop codon and a truncated protein. In some embodiments, a substitution that produces a non-functional and/or truncated protein may be about 2 to about 20 nucleotides near the 5′ end of the MIXTA transcription factor gene. As described herein, the endogenous MIXTA transcription factor gene may be present in a Rubus plant or plant cell in multiple copies of and each copy may comprise at least one non-natural mutation. In some embodiments, the Rubus plant is, for example, a blackberry plant, a black raspberry plant or a raspberry plant. In some embodiments, the Rubus plant is a blackberry plant, which comprises four copies of the endogenous MIXTA transcription factor gene, and each copy in the blackberry plant or plant part thereof is edited and comprises a mutation/edit as described herein. In some embodiments, the Rubus plant is a blackberry plant, which comprises six copies of the endogenous MIXTA transcription factor gene, and each copy in the blackberry plant or plant part thereof is edited and comprises a mutation/edit as described herein. In some embodiments, the Rubus plant is a blackberry plant, which comprises eight copies of the endogenous MIXTA transcription factor gene, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene.

A non-natural mutation in an endogenous gene encoding a MIXTA transcription factor that provides Rubus plants (e.g., blackberry, black raspberry, raspberry) with reduced or no thorns or prickles may be a dominant negative mutation or a null mutation.

In some embodiments, a mutation in an endogenous MIXTA transcription factor gene may be made following cleavage by an editing system that comprises a nuclease and a DNA-binding domain that binds to a target site within a target nucleic acid comprising the nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83, or encoding a polypeptide comprising the sequence of any one of the amino acid sequences of SEQ ID NOs: 57-62, 71-76, 90-92, 94, 96, 98, or 100 or a sequence having at least at least 94% identity to the amino acid sequence of SEQ ID NO:57. In some embodiments, the nuclease cleaves the endogenous MIXTA transcription factor gene and a mutation is introduced into the endogenous MIXTA transcription factor gene. In some embodiments, the editing system introduces a deletion or a substitution of one or more nucleotides (e.g., 1 to about 25 (consecutive or nonconsecutive) nucleotides) into the endogenous MIXTA transcription factor gene.

Further provided herein are guide nucleic acids (e.g., gRNA, gDNA, crRNA, crDNA) that bind to a target site in a MIXTA transcription factor gene, wherein the endogenous MIXTA transcription factor gene: (i) encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57, or (ii) comprises a nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83. In some embodiments, the guide nucleic acid comprises a spacer having the nucleotide sequence of any one of SEQ ID NOs:53-56 or 109-117. As described herein, the endogenous MIXTA transcription factor gene may be present in the Rubus plant or plant cell (e.g., blackberry, black raspberry, or raspberry) in multiple copies (e.g., 2, 4, 5, 6, 8, 10, 12, or 14, or more copies) and the same or different spacers may be used to edit each copy of the endogenous gene so that each copy comprises at least one mutation as described herein. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene. In some embodiments, the guide nucleic acids can be used to introduce a deletion or a substitution of one or more nucleotides (e.g., 1 to about 25 nucleotides (consecutive or nonconsecutive)) into the endogenous MIXTA transcription factor gene.

In some embodiments, a system is provided comprising a guide nucleic acid comprising a spacer having the nucleotide sequence of any one of SEQ ID NOs:53-56 or SEQ ID NOs:109-117 and a CRISPR-Cas effector protein that associates with the guide nucleic acid. In some embodiments, the system may further comprise a tracr nucleic acid that associates with the guide nucleic acid and a CRISPR-Cas effector protein, optionally wherein the tracr nucleic acid and the guide nucleic acid are covalently linked. In some embodiments, the system introduces a deletion or a substitution of one or more nucleotides (e.g., 1 to about 25 (consecutive or nonconsecutive) nucleotides) into the endogenous gene.

The invention further provides a gene editing system comprising a CRISPR-Cas effector protein in association with a guide nucleic acid and the guide nucleic acid comprises a spacer sequence that binds to a MIXTA transcription factor gene that comprises a nucleotide sequence of any one of SEQ ID NOs: 63-70, 77, 84-89, 93, 95, 97, or 99 or a nucleotide sequence having at least 95% identity to the nucleotide sequence of SEQ ID NO:78 or SEQ ID NO:83, or encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57. In some embodiments, a spacer sequence of the guide nucleic acid may comprise the nucleotide sequence of any one of SEQ ID NOs:53-56 or 109-117. In some embodiments, the gene editing system may further comprise a tracr nucleic acid that associates with the guide nucleic acid and a CRISPR-Cas effector protein, optionally wherein the tracr nucleic acid and the guide nucleic acid are covalently linked. In some embodiments, the gene editing system introduces a deletion or a substitution of one or more nucleotides (e.g., 1 to about 25 (consecutive or nonconsecutive) nucleotides) into an endogenous MIXTA transcription factor gene.

The present invention further provides a complex comprising a CRISPR-Cas effector protein comprising a cleavage domain and a guide nucleic acid, wherein the guide nucleic acid binds to a target site in an endogenous MIXTA transcription factor gene, wherein the MIXTA transcription factor gene comprises a nucleotide sequence of any one of SEQ ID NOs:63-70, 77, 84-89, 93, 95, 97, or 99 or a nucleotide sequence having at least 95% identity to the nucleotide sequence of SEQ ID NO:78 or SEQ ID NO:83, or encodes a polypeptide comprising the sequence of any one of the amino acid sequences of SEQ ID NOs:57-62, 71, 72, 94, 96, 98, or 100 or a sequence having at least at least 94% identity to any one of the amino acid sequences of SEQ ID NO:57, wherein the cleavage domain cleaves a target strand in the MIXTA transcription factor gene. In some embodiments, the complex introduces a deletion or a substitution of one or more nucleotides (e.g., 1 to about 25 (consecutive or nonconsecutive) nucleotides) into an endogenous MIXTA transcription factor gene.

In some embodiments, expression cassettes are provided that comprise (a) a polynucleotide encoding CRISPR-Cas effector protein comprising a cleavage domain and (b) a guide nucleic acid that binds to a target site in an endogenous MIXTA transcription factor gene, wherein the guide nucleic acid comprises a spacer sequence that is complementary to and binds: (i) to a portion of the nucleotide sequence of SEQ ID NO:77 or 84-89 or a nucleotide sequence having at least 95% identity to the nucleotide sequence of SEQ ID NO:78 or SEQ ID NO:83 or (ii) to a portion of a nucleotide sequence encoding any one of the amino acid sequences of SEQ ID NOs:73-76 or 90-92.

Also provided herein are nucleic acids encoding a null allele of a MIXTA transcription factor, wherein the null allele when present in a Rubus plant or plant part results in reduced or no thorns/prickles. Additionally provided are nucleic acids encoding a dominant negative mutation of a MIXTA transcription factor, wherein the dominant negative mutation when present in a Rubus plant or plant part results in reduced or no thorns/prickles.

Nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids (e.g., endogenous MIXTA transcription factors of Rubus plants) and/or their expression.

Any Rubus plant comprising an endogenous MIXTA transcription factor that is capable of regulating thorn/prickle development in a Rubus plant may be modified (e.g., mutated, e.g., base edited, cleaved, nicked, etc.) as described herein (e.g., using the polypeptides, polynucleotides, RNPs, nucleic acid constructs, expression cassettes, and/or vectors of the invention) to reduce or eliminate thorn or prickle production in the plant. A Rubus plant having reduced thorns or prickles may have a reduction in thorns or prickles of about 10% to about 100% (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or any range or value therein) as compared to a Rubus plant or part thereof that does not comprise the mutated endogenous MIXTA transcription factor. In some embodiments, a Rubus plant having a reduction in thorns/prickles may be devoid of thorns or prickles. In some embodiments, a Rubus plant of the invention comprise stems that are about 10% to 100% (e.g., about 10%, 20%, 30%, 40%, 50% to about 80%, 90%, 95%, 98%, 99%, or 100%; e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%) devoid of prickles/thorns as compared to the of a stem of a control Rubus plant (e.g., the same Rubus species/cultivar/strain that does not comprise the mutated endogenous MIXTA transcription factor). In some embodiments, the gene encoding the MIXTA transcription factor in a Rubus plant may be present in at least two copies (e.g., 2, 4, 6, 8, 10, 12, or 14, or more copies), wherein each copy comprises a mutation as described herein (e.g., homozygous for the mutation). In some embodiments, the Rubus plant is, for example, a blackberry plant, a black raspberry plant or a raspberry plant. In some embodiments, the Rubus plant is a blackberry plant, which comprises two copies of the endogenous MIXTA transcription factor gene, and each copy in the blackberry plant or plant part thereof is edited and comprises a mutation/edit as described herein. In some embodiments, the Rubus plant is a blackberry plant, which comprises four copies of the endogenous MIXTA transcription factor gene, and each copy in the blackberry plant or plant part thereof is edited and comprises a mutation/edit as described herein. In some embodiments, the Rubus plant is a blackberry plant, which comprises six copies of the endogenous MIXTA transcription factor gene, and each copy in the blackberry plant or plant part thereof is edited and comprises a mutation/edit as described herein. In some embodiments, the Rubus plant is a blackberry plant, which comprises eight copies of the endogenous MIXTA transcription factor gene, and each copy in the blackberry plant or plant part thereof is edited and comprises a mutation/edit as described herein. When multiple copies of the MIXTA gene are present in a genome the edit may be introduced into each copy of a MIXTA gene by editing as described herein, or by editing one or more copies of a MIXTA gene in a plant as described herein and then selfing the plant comprising the edit in one or more copies to produce a plant comprising an edit in each copy of the MIXTA gene (e.g., homozygous for the mutation). In some embodiments, an edit useful with this invention can be a deletion of about 1 to about 25 nucleotides (1, 2, 3, 4, 5, 6, 7, 8, 9, 10 to about 11, 12, 13, 14, 15, 16, 17, 18, 19, 20, 21, 22, 23, 24, 25 or a substitution of at least 2 nucleotides (e.g., 2, 3, 4, 5, 6, 7, 8, 9, or 10 or more), which results in a non-functional polypeptide, e.g., truncated polypeptide, a null allele.

In some embodiments, a Rubus plant or part thereof (e.g., a blackberry plant, a black raspberry plant or a red raspberry plant) comprising a mutation in an endogenous MIXTA transcription factor gene may comprise mutations (e.g., one or more non-natural mutations) in one or more endogenous genes encoding polypeptides of interest including, but not limited to, AG clade MADS-box transcription factor genes. AG clade MADS-box transcription factor genes include, for example, AGAMOUS (AG), SHATTERPROOF 1 (SHP1), SHATTERPROOF 2 (SHP2), and/or SEEDSTICK (STK). In some embodiments, a Rubus plant or part thereof comprising a mutation in an endogenous MIXTA transcription factor gene may also comprise a mutation in an endogenous gene encoding SEEDSTICK (STK), thereby providing a Rubus plant that has reduced thorns/prickles and is seedless or has reduced seediness, reduced seed lignin content, reduced seed endocarp formation, and/or a smoother seed surface.

A mutation in an endogenous AG clade MADS-box transcription factor gene of a Rubus plant or part thereof can result in production of seedless fruits or fruits with reduced seediness. Seedless and/or reduced seediness fruit is defined as a fruit that does not have a seed or a pit/drupe (drupeless) or, seeded fruits wherein the seed is altered compared to a wild type fruit of the same plant species due to reduced seed size or reduced seed lignin content or reduced endocarp formation. Reduced seediness is defined as the following: reduced lignin content as compared to wild type (lignin/milligram protein-free cell wall in endocarp and/or seed coat; reduced seed size as compared to wild type (volume of seed and/or drupe); and/or smoother seed/drupe surface as compared to wild type (seed/drupe surface area/volume).

A non-natural mutation in an AG clade MADS-box transcription factor such as AGAMOUS (AG), SHATTERPROOF 1 (SHP1), SHATTERPROOF 2 (SHP2), and/or SEEDSTICK (STK) can result in a dominant-negative allele, semi-dominant allele, weak loss of function allele, or a hypomorphic mutation and a plant that is seedless or has reduced seediness. In some embodiments, the mutation is a dominant-negative allele or a weak loss of function allele. In some embodiments, the mutation is a weak loss of function allele, or a hypomorphic mutation.

In some embodiments, the endogenous gene encoding an AG clade MADS-box transcription factor is capable of regulating seed production. In some embodiments, a Rubus plant comprising the at least one non-natural mutation in an endogenous gene encoding an AG clade MADS-box transcription factor exhibits altered fruit development, optionally wherein the endogenous gene encoding an AG clade MADS-box transcription factor is a SEEDSTICK (STK) transcription factor gene. In some embodiments, altered fruit development comprises a phenotype including, but not limited to, seedlessness (e.g., no seed or a reduced number of seeds), reduced seediness, reduced seed lignin content, reduced seed endocarp formation, or smoother seed surface. A non-natural mutation in an endogenous gene encoding an AG clade MADS-box transcription factor to provide an altered seed development in a Rubus plant can be a base substitution, a base deletion and/or a base insertion. In some embodiments, a base substitution to an A, a T, a G, or a C. In some embodiments, the at least one non-natural mutation is a substitution of at least one base pair (e.g., 1, 2, 3, 4, or 5 base pairs). In some embodiments, the at least one non-natural mutation results in a deletion of the 3′ end of the gene and a polypeptide having a C-terminal truncation or results in a deletion of the 5′ end of the gene and a polypeptide having an N-terminal truncation. The generation of mutations in endogenous genes encoding AG clade MADS-box transcription factors useful with this invention are described in detail in International Patent Application Publication WO/2020/252167 (on Dec. 17, 2020).

In some embodiments, specific motifs/domains as described in WO/2020/252167 may be modified in a Rubus plant or part thereof produced according to methods of this invention to produce a Rubus plant or part thereof having reduced thorns or prickles that is seedless and/or has reduced seediness, reduced seed lignin content, reduced seed endocarp formation, or a smoother seed surface as compared to a Rubus plant not comprising the non-natural mutation in the endogenous MIXTA gene and the non-natural mutation in the endogenous STK gene. In some embodiments, the at least one non-natural mutation in an STK gene results in a substitution in an amino acid residue as described in WO/2020/252167. In some embodiments, the at least one non-natural mutation in an AG clade MADS-box transcription factor that is a STK gene is an amino acid substitution at a conserved arginine in the STK gene as described in WO/2020/252167.

In some embodiments, the at least one non-natural mutation is in the C-terminal domain of the polypeptide encoded by the endogenous gene encoding an AG clade MADS-box transcription factor, optionally in the C-terminal domain of a polypeptide encoded by a STK gene. In some embodiments, at least one non-natural mutation in an AG clade MADS-box transcription factor results in a deletion of at least one amino acid or at least two or more consecutive amino acid. In some embodiments, the non-natural mutation is a deletion of at least 4 consecutive base pairs to about 150 consecutive base pairs from a STK gene, wherein the deletion results in a C-terminal truncation comprising a truncation of at least 1 amino acid residue to about 50 consecutive amino acid residues.

In some embodiments, a deletion is an in-frame deletion or an out-of-frame deletion, optionally wherein the in-frame mutation or the out-of-frame mutation results in a non-natural/premature stop codon and a C-terminal truncation of one amino acid residue or two or more consecutive amino acid of an endogenous gene encoding an AG clade MADS-box transcription factor, optionally in an endogenous gene encoding an STK gene. Such deletions can alter the reading frame resulting in premature termination of translation, e.g., a premature stop codon a truncation of the polypeptide. In some embodiments, the at least one non-natural mutation in the C-terminal domain is a C-terminal truncation, optionally wherein at least the last 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 consecutive amino acids of the C-terminus of an STK polypeptide are truncated. Such a truncation results in the removal of a conserved arginine in the C-terminus of the STK polypeptide as described in WO/2020/252167, optionally wherein residues surrounding the conserved arginine are removed.

In some embodiments, a Rubus plant is provided that comprises at least one non-natural mutation in an endogenous gene encoding a MIXTA transcription factor and at least one non-natural mutation in an endogenous AG clade MADS-box transcription factor gene, the Rubus plant having reduced thorns and/or prickles and altered fruit development, wherein the at least one non-natural mutation in the endogenous gene encoding a MIXTA transcription factor is as described herein, and the at least one non-natural mutation in an endogenous AG clade MADS-box transcription factor gene is a base substitution, insertion or a deletion that is introduced using an editing system that comprises a nucleic acid binding domain that binds to a target site in the AG clade MADS-box transcription factor gene, optionally wherein the AG clade MADS-box transcription factor gene is a SEEDSTICK (STK) gene, the STK gene as described in WO/2020/252167.

The invention further provides a Rubus plant or part thereof comprising at least one non-natural mutation in an endogenous gene encoding a MIXTA transcription factor as described herein and at least one non-natural mutation in an endogenous SEEDSTICK (STK) gene, wherein the endogenous STK gene (a) comprises a nucleic acid sequence as described in WO/2020/252167; or (b) encodes an amino acid sequence as described in WO/2020/252167; or a sequence comprising a region as described in WO/2020/252167, wherein the plant comprising the mutated STK gene and mutated MIXTA gene exhibits altered fruit development and reduced thorns and/or prickles as compared to a Rubus plant not comprising the non-natural mutation in the endogenous MIXTA gene and the non-natural mutation in the endogenous STK gene.

A Rubus plant and/or plant part that may be modified as described herein may be any Rubus species, variety and/or cultivar. Non-limiting examples of Rubus plants that may be modified as described herein include blackberry, black raspberry or raspberry (e.g., red raspberry). Rubus plants useful with the invention can include, but are not limited to, Rubus occidentalis L., Rubus pergratus Blanch., Rubus oklahomus L.H. Bailey Rubus originalis L.H. Bailey, Rubus ortivus (L.H. Bailey) L.H. Bailey, Rubus parcifrondifer L.H. Bailey, Rubus odoratus L., Rubus parvifohus L., Rubus pedatus Sm., and Rubus phoenicolasius Maxim. In some embodiments, the Rubus plant comprises two or more copies (e.g., 2, 4, 6, 8, 10, 12, or 14, or more copies) of an endogenous MIXTA gene. For example, blackberry may comprise two or more copies of an endogenous MIXTA gene. In some embodiments, when a Rubus plant such as blackberry comprises two or more copies of the endogenous MIXTA gene, each copy may comprise an edit/non-natural mutation as described herein. When multiple copies of the MIXTA gene are present in a genome the edit may be introduced into each copy of a MIXTA gene by editing as described herein, or by editing one or more copies of a MIXTA gene in a plant as described herein and then selfing the plant comprising the edit in one or more copies to produce a plant comprising an edit in each copy of the MIXTA gene. When multiple copies of the MIXTA gene are present in a genome the edit may be introduced into each copy of a MIXTA gene by editing as described herein, or by editing one or more copies of the MIXTA gene in a plant as described herein and then selfing the plant comprising the edit in one or more copies to produce a plant comprising an edit in each copy of the MIXTA gene.

The term “plant part,” as used herein, includes but is not limited to reproductive tissues (e.g., petals, sepals, stamens, pistils, receptacles, anthers, pollen, flowers, fruits, flower bud, ovules, seeds, and embryos); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus. The term “stem” as used herein refers the above ground structural axis of the plant consisting of both nodes (e.g., leaves and flowers) and internodes (e.g., connecting material between nodes).

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

An editing system useful with this invention can be any site-specific (sequence-specific) genome editing system now known or later developed, which system can introduce mutations in target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing a MIXTA transcription factor may comprise contacting a target nucleic acid (e.g., a nucleic acid encoding a MIXTA transcription factor) with a base-editing fusion protein (e.g., a sequence specific DNA binding protein (e.g., a CRISPR-Cas effector protein or domain) fused to a deaminase domain (e.g., an adenine deaminase and/or a cytosine deaminase) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific DNA binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a MIXTA transcription factor may comprise contacting a target nucleic acid (e.g., a nucleic acid encoding a MIXTA transcription factor) with a sequence-specific DNA binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific DNA binding fusion protein to the target nucleic acid and the sequence-specific DNA binding fusion protein is capable of recruiting the deaminase fusion protein to the target nucleic acid via the peptide tag-affinity polypeptide interaction, thereby editing a locus within the target nucleic acid. In some embodiments, the sequence-specific DNA binding fusion protein may be fused to the affinity polypeptide that binds the peptide tag and the deaminase may be fuse to the peptide tag, thereby recruiting the deaminase to the sequence-specific DNA binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific DNA binding fusion proteins, deaminase fusion proteins and guides may be provided as ribonucleoproteins (RNPs).

In some embodiments, methods such as prime editing may be used to generate a mutation in an endogenous MIXTA transcription factor gene. In prime editing, RNA-dependent DNA polymerase (reverse transcriptase, RT) and reverse transcriptase templates (RT template) are used in combination with sequence specific DNA binding domains that confer the ability to recognize and bind the target in a sequence-specific manner, and which can also cause a nick of the PAM-containing strand within the target. The DNA binding domain may be a CRISPR-Cas effector protein and in this case, the CRISPR array or guide RNA may be an extended guide that comprises an extended portion comprising a primer binding site (PSB) and the edit to be incorporated into the genome (the template). Similar to base editing, prime editing can take advantageous of the various methods of recruiting proteins for use in the editing to the target site, such methods including both non-covalent and covalent interactions between the proteins and nucleic acids used in the selected process of genome editing.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific DNA binding domain may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain, e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophiles), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO:79 and SEQ ID NO:80.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat or a mouse. Thus, in some embodiments, a cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27 or SEQ ID NO:28. In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27 or SEQ ID NO:28). In some embodiments, a polynucleotide encoding a cytosine deaminase may be codon optimized for expression in a plant and the codon optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be codon optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be codon optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:35 or a polypeptide having about 70% to about 99.5% sequence identity to the amino acid sequence of SEQ ID NO:35 (e.g., at least 80%, at least 85%, at least 90%, at least 92%, at least 95%, at least 96%, at least 97%, at least 98%, at least 99%, or at least 99.5% identical to the amino acid sequence of SEQ ID NO:35). For example, in some embodiments, a UGI domain may comprise a fragment of the amino acid sequence of SEQ ID NO:35 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:35. In some embodiments, a UGI domain may be a variant of a known UGI (e.g., SEQ ID NO:35) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be codon optimized for expression in a plant (e.g., a plant) and the codon optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-34 (e.g., SEQ ID NOs: 31, 32, 33, or 34). In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35 (Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., consecutive nucleotides of the nucleotide sequences of any one of SEQ ID NOs:63-70, 77, 84-89, 93, 95, 97, or 99 or a sequence having at least 95% identity to the nucleotide sequence of SEQ ID NO:78 or SEQ ID NO:83 or a nucleotide sequence encoding a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57). In some embodiments, a spacer sequence may include, but is not limited to, the nucleotide sequences of any one of SEQ ID NOs:53-56 or 109-117. The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer (SEQ ID NO: 36)
            ||||||||||||||||||
3′AAANNNNNNNNNNNNNNNNNNN-5′ Target strand (SEQ ID NO: 37)
            ||||||||||||||||||
5′TTTNNNNNNNNNNNNNNNNNNN-3′ Non-target strand (SEQ ID NO: 38)
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types, and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g, one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific DNA binding domains, CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RCD-His affinity tag, a FLAG octapeptide, a strep tag or strep tag I, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases).

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs, e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Corn RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihydrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been codon optimized for expression in a plant.

Further provided herein are cells comprising one or more polynucleotides, guide nucleic acids, nucleic acid constructs, expression cassettes or vectors of the invention.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. Identification of MIXTA Genes

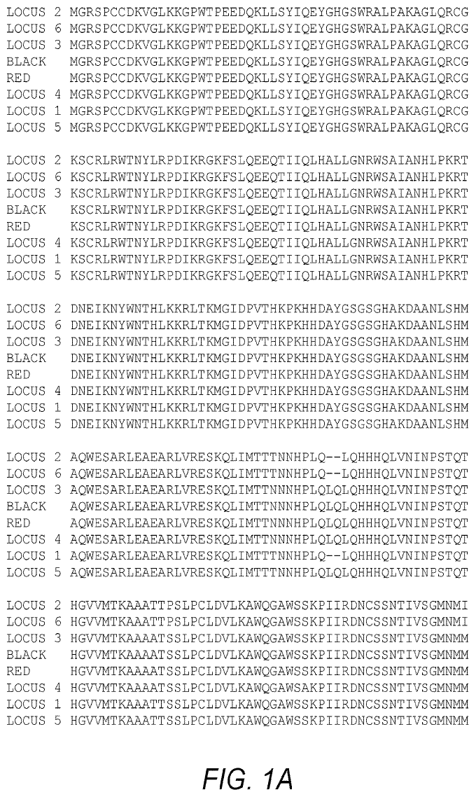

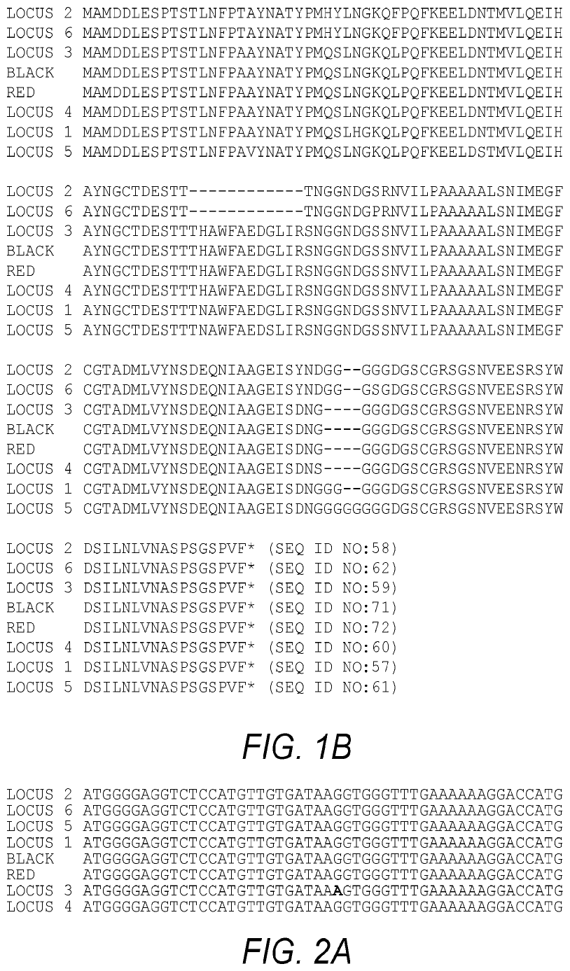

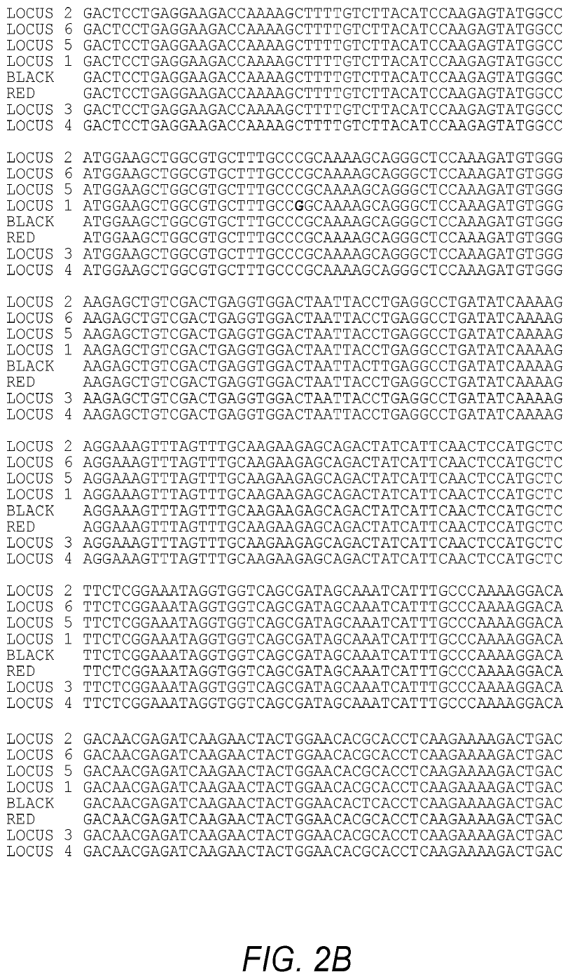

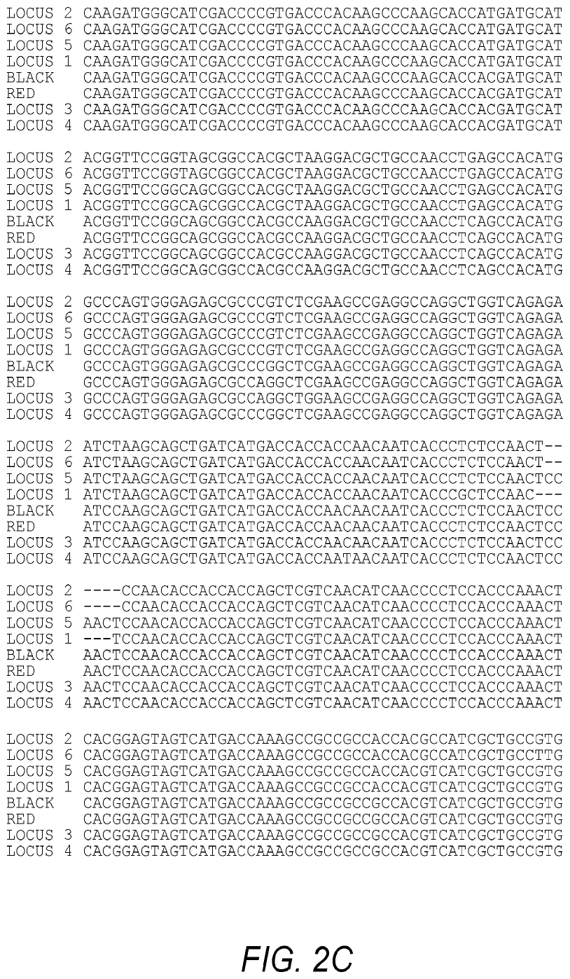

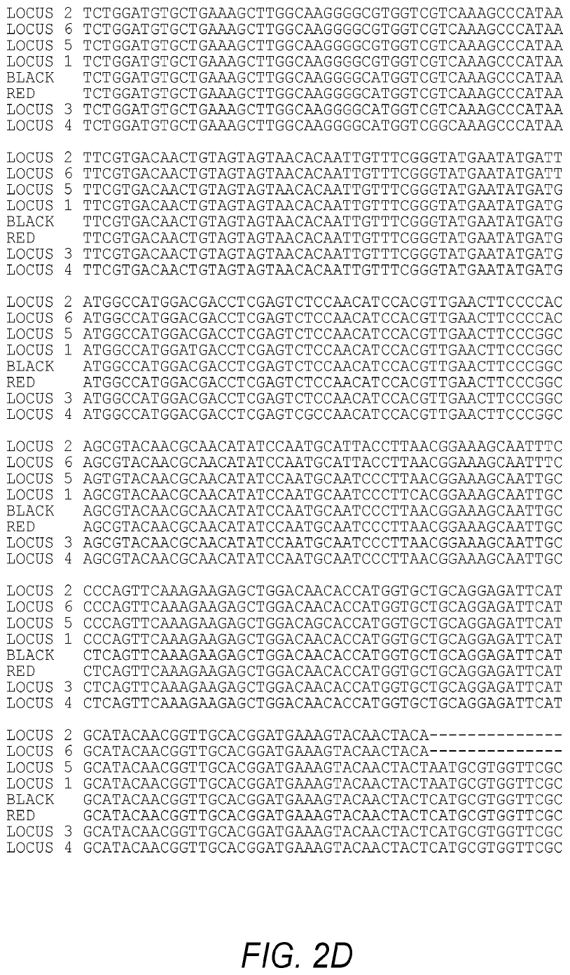

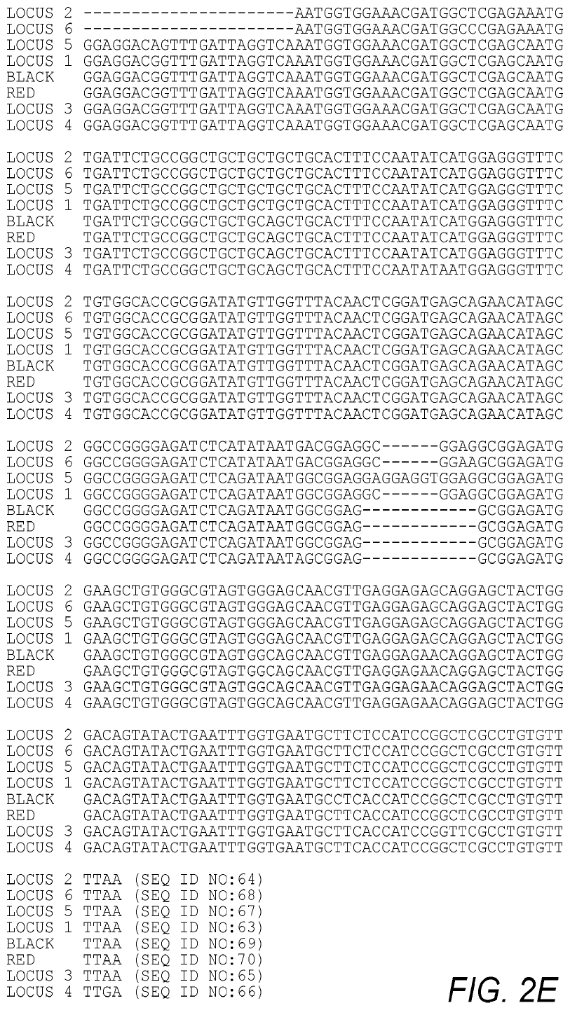

Blackberry MIXTA genes were identified and verified (SEQ ID NOs:63-68, 93, 95, 97, 99). Orthologous MIXTA genes in red raspberry and black raspberry were also identified and verified (SEQ ID NOs:69-70). An alignment of the blackberry, red raspberry and blackberry sequences are provided in FIGS. 1A-1B and FIGS. 2A-2D showing that these sequences share significant sequence identity. Regions within the MIXTA nucleic acids were targeted including those represented by SEQ ID NOs:77, 78, or 83-89.

Also identified is a MIXTA gene from strawberry (SEQ ID NO:121). As discussed previously, it is believed that glandular trichomes are involved in the development of prickles in Rubus spp. Wild strawberry (Fragaria vesca) provides a model system for studying glandular trichome development. In particular, the rapid growth cycle of strawberry provides the ability to more quickly evaluate the effect of MIXTA mutations generated as described herein on the development of glandular trichomes. Wild strawberry is also a diploid in contrast to Rubus plants of which many are polyploidy. Consequently, generating plants that are homozygous for a MIXTA mutation may be quicker in wild strawberry.

### Example 2. Construction of Base Editing Vectors

Target sites for site-specific mutation(s) in the candidate MIXTA transcription factors were selected by ability to interfere with gene function through introduction of a STOP codon or change in a conserved amino acid in the MYB domain. Base editing vectors for the target nucleic acid/target site in the candidate MIXTA transcription factor identified in plants of interest are constructed for the transformation of the base-editing vectors into the plant of interest including, but not limited to: Rubus spp., black raspberry, blackberry, red raspberry, strawberry, and wild strawberry.

Cytosine Base Editor (CBE) expression vectors and Adenine Base Editor (ABE) expression vectors are generated to introduce into candidate gene(s) (e.g., MIXTA transcription factor genes and their orthologs in plants) mutations that result in thornlessness or pricklessness or reduced thorns and/or prickles (or reduced glandular trichomes in strawberry). By using genetic tools to introduce mutations to modify MIXTA genes as described herein, gRNAs will be designed accordingly to introduce deletions and substitutions, which produce truncated and/or non-functional polypeptides. The genetic modification introduced by the base-editing techniques will results in amino acid substitution, addition, deletion, and/or truncation. Introduction of premature stop codons, as a result of deletions, substitutions, or additions, will produce truncations in the encoded polypeptide; that is, all of the amino acid sequence downstream of the newly-introduced stop codon will be deleted/truncated accordingly.

Targeting Cutter Vectors to Make a C-Terminal Truncation/Deletion

Cas12a expression vectors will be generated to introduce into candidate gene(s) (e.g. MIXTA transcription factor genes and their orthologs in plants) mutations that cause reduced thorns and/or prickles or thornlessness and/or pricklessness. Guide RNAs (gRNAs) are designed to introduce base pair deletions or substitutions. The genetic modification introduced by the base pair deletions will result in amino acid substitutions and/or amino acid deletions and/or truncation of the encoded polypeptide.

1. Construction of Cytosine Base Editor (CBE) Expression Vector

Cytosine deaminases (CDs) typically deaminate cytosines at specific sites in single stranded DNA so that the deamination of cytosine (C) is catalyzed by cytidine deaminases and results in uracil (U), which has the base-pairing properties of thymine (T). In this example, CD is fused to nuclease-deficient type II CRISPR/Cas9 to achieve RNA-guided cytosine deamination on genomic DNA and CD edits a non-targeted DNA strand displaced by the binding of a Cas9-guide RNA complex to a targeted DNA strand. To induce single-nucleotide substitution, either catalytically dead/deficient Cas9 (dCas9) whose nuclease activity is lost or Cas9 variant having nickase activity (nCas9) is used for Cas9-CD fusion protein. While dCas9 or nCas9 is bound to its target via guide RNA and form the bubble between the guide RNA and its complementary DNA, the CD enzyme will then act on the cytosine in the accessible single stranded DNA in the mismatching bubble. When nCas9 is utilized, the Cas9 nickase will nick the non-targeted DNA strand and facilitate subsequent conversion of the corresponding guanine to Adenine on the nontargeted DNA strand.

Additional CRISPR-Cas systems may be used. For example, the CRISPR nuclease-deficient systems such as Cpf1 nickase, nuclease-deficient C2c1 or nuclease-deficient Cms1 can be used to nick the non-targeted DNA strand. Thus, a nuclease deficient Cas9, CasX, CasY, Cpf1, C2c1, C2c2, C2c3 or Cms1 can be used for single base substitution along with base-editing enzymes cytidine deaminase and/or adenosine deaminase. For the CBE expression vector construction, the APOBEC1, XTEN, nCas9(D10A) or dCas9 (D10A and H840A), and UGI sequences are codon-optimized for plants of interest. A uracil glycosylase inhibitor (UGI) can be fused to the nCas9 or dCas9 to inhibit uracil-DNA glycosylase base-excision repair enzyme and increase the efficiency of base editing.

Designs for the CBE expression vector:
nCas9, dCas9, Cpf1 nickase, nuclease-deficient C2c1 or nuclease-deficient Cms1 is fused to CD on either the N- or C-terminal end.
Nuclear Localization Sequence (NLS) can be tagged to nCas9, nCas9, dCas9, Cpf1 nickase, nuclease-deficient C2c1 or nuclease-deficient Cms1.
A variety of linkers are used including flexible linkers such as XTEN or less flexible linkers.
A uracil glycosylase inhibitor (UGI) can be linked to the nCas9, dCas9, Cpf1 nickase, nuclease-deficient C2c1 or nuclease-deficient Cms1.
The guide RNA scaffold can be inserted in the CBE expression vector or be expressed in a separate guide RNA expression vector.

2. Construction of Adenine Base Editor (ABE) Expression Vector

Adenosine deaminases (ADs) typically deaminate adenine in a deoxyadenosine residue of DNA at specific sites in single stranded DNA so that the deamination of adenosine (A) is catalyzed by adenosine deaminases and results in Inosine, which base pairs like guanine (G) in the context of DNA and has the base-pairing properties of Guanine (G). In this example, AD is fused to nuclease-deficient type II CRISPR/Cas9 to achieve RNA-guided cytosine deamination on genomic DNA and AD edits a non-targeted DNA strand displaced by the binding of a Cas9-guide RNA complex to a targeted DNA strand. To induce single-nucleotide substitution, either catalytically dead/deficient Cas9 (dCas9) whose nuclease activity is lost or Cas9 variant having nickase activity (nCas9) is used for Cas9-AD fusion protein. While dCas9 or nCas9 is bound to its target via guide RNA and form the bubble between the guide RNA and its complementary DNA, the AD enzyme will then act on the adenosine in the accessible single stranded DNA in the mismatching bubble. When nCas9 is utilized, the Cas9 nickase will nick the non-targeted DNA strand and facilitate subsequent conversion of the corresponding Thymine to Cytosine on the nontargeted DNA strand.

Additional CRISPR-Cas systems may be used. For example, the CRISPR nuclease-deficient systems such as Cpf1 nickase, nuclease-deficient C2c1 or nuclease-deficient Cms1 can be used to nick the non-targeted DNA strand. For example, the CRISPR nuclease-deficient systems such as Cpf1 nickase, nuclease-deficient C2c1 or nuclease-deficient Cms1 can be used to nick the non-targeted DNA strand. Thus, a nuclease deficient Cas9, CasX, CasY, Cpf1, C2c1, C2c2, C2c3 or Cms1 can be used for single base substitution along with base-editing enzymes cytidine deaminase and/or adenosine deaminase. For the ABE expression vector construction, the E. coli TadA (ecTadA) including truncations/mutations of adenosine deaminase, human ADAR including hADAR1, hADAR2, hADAR3 and mutated versions thereof, XTEN, nCas9(D10A) or dCas9 (D10A and H840A), and UGI sequences are codon-optimized for plants of interest. A uracil glycosylase inhibitor (UGI) can be fused to the nCas9 or dCas9 to inhibit uracil-DNA glycosylase base-excision repair enzyme and increase the efficiency of base editing.

Designs for the ABE Expression Vector:
nCas9, dCas9, Cpf1 nickase, nuclease-deficient C2c1, C2c2, C2c3 or nuclease-deficient Cms1 is fused to CD on either the N- or C-terminal end.
Nuclear Localization Sequence (NLS) can be tagged to nCas9, nCas9, dCas9, Cpf1 nickase, nuclease-deficient C2c1 or nuclease-deficient Cms1.
A variety of linkers are used including flexible linkers such as XTEN or less flexible linkers.
A uracil glycosylase inhibitor (UGI) can be linked to the nCas9, dCas9, Cpf1 nickase, nuclease-deficient C2c1, C2c1, C2c2, C2c3 or nuclease-deficient Cms1.
The guide RNA scaffold can be inserted in the CBE expression vector or be expressed in a separate guide RNA expression vector.

3. Designs for gRNA Scaffold

The guide system depends upon the CRISPR system utilized. If the CRISPR system utilizes Cpf1, the guide is a natural “single” guide, but with Cas9 it can be single, or dual/native with separate crRNA tracrRNA. Base editing vectors targeting MIXTA genes will be further constructed by inserting guide RNAs that contain a spacer that is complementary to a target site in the regions of the MIXTA genes as described herein including but not limited to SEQ ID NOs:77, 78, or 83-89 (portions of MIXTA genes (SEQ ID NOs:63-70, 93, 95, 97, or 99)). Once the guide RNA leads the CRISPR system to the target sites in the MIXTA nucleic acid, cytosine located in the base-editing window, which is 4-8 nucleotides (nt) distal to PAM is deaminated and converted into Uracil, which is used as Thymine in a CBE system. Alternatively using an adenosine base editor, the guide RNA leads the Cas9/AD fusion protein to the target sites in in the MIXTA nucleic acid, adenine located in the base-editing window, which is 4-8 nt distal to PAM will be deaminated and converted into Inosine that is used as Guanine by polymerase in ABE system.

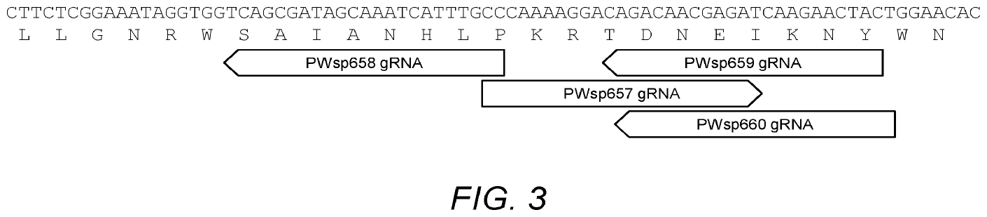

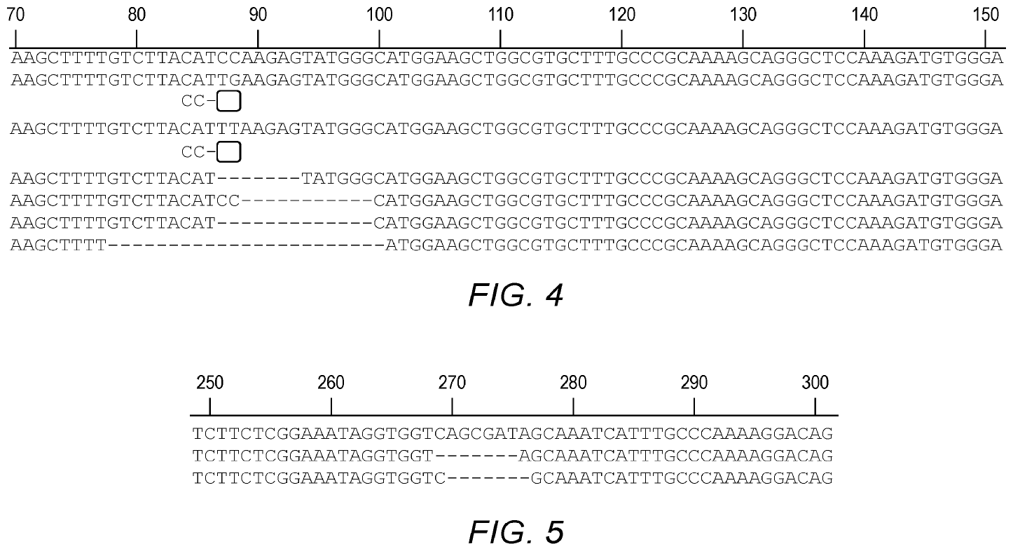

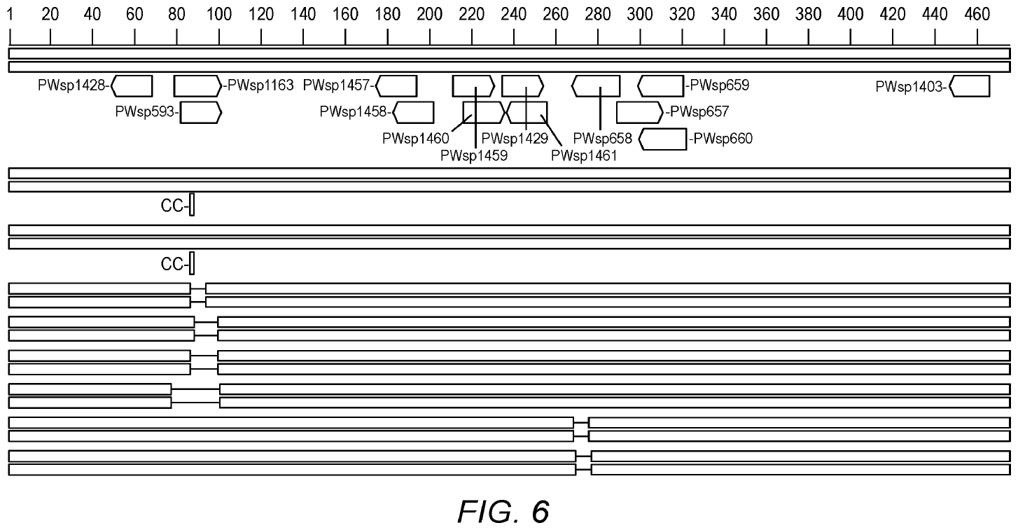

FIG. 3 and FIG. 6 illustrate the locations of four example guide RNAs (SEQ ID NOs:53-56), and thirteen example guide RNAs (SEQ ID NOs:53-56 and SEQ ID NOs:109-117) designed to target the regions of the MIXTA genes as described herein (see, e.g., SEQ ID NOs:77, 78, or 83-89). These regions are shared among the MIXTA genes identified in Rubus spp. and make possible the targeting of a wide variety of species in the genus Rubus using the methods described herein.

Appropriate PAM sequence is used for gRNA design and depend on the Cas system used. The source/type of Cas9 protein as Cas9 variants have different PAM requirement for target recognition. PAM sites in a gene of interest can be determined. As an example, the PAM site recognized by Cas9 is NGG, Cpf1 (Cas12a) recognizes TTTN and C2c1/Cms1 recognizes TTN.

### Example 3. Transformation with Base-Editing Vectors into Plants of Interest

The base-editing vectors discussed in Example 2 are transformed into Rubus plants including black raspberry, blackberry, red raspberry, and into strawberry and wild strawberry (Fragaria vesca). The disclosure teaches all types of transformation methods, including using agrobacterium-mediated protocols that are known in the art and/or developed by the inventors, as well as biolistic transformation methods. Tissue culture and regeneration of transformed plants will be performed accordingly.

### Example 4. Targeting of MIXTA Genes in Plants

Blackberry lines 15.025-01, 15.039-04, and 17.006-13 (part of the genus Rubus subgenus Rubus (formerly subgenus Eubatus), Rosaceae, the black raspberry line 13.013-11 (a cultivar of Rubus occidentalis) and a diploid strawberry (Hawaii-4 Fragaria vesca) were transformed with the CRIPSR constructs as described herein and thorn, prickle or glandular trichome development examined.

MIXTA transcription factor genes were targeted as described herein to generate prickleless blackberry plants (in lines 15.025-01, 15.039-04 and 17.006-13), prickleless black raspberry plants and strawberry plants (Hawaii-4) lacking glandular trichomes.

Base editing vectors targeting the MIXTA gene were constructed by inserting single guide RNA fragments that contain one target single site with the aim to insert a stop codon into the MIXTA locus (via, for example, base deletions or substitutions) or to substitute a conserved amino acid in the MYB domain of the MIXTA locus for an alternative amino acid residue. Plasmid vectors containing the PWsp1403 spacer were utilized for these experiments.

TABLE 1
Blackberry lines, s pacer sequences and edits

| | | | | | Change |
|---|---|---|---|---|---|
| Line | Spacer | Sequence | PAM | Enzyme | observed |
| 15.025-01 | PWsp1428 | TGGTCTTCCTCAGGAGTCCA | TGG | Cas9CBE | E > K myb |
| 15.039-04 | | SEQ ID NO: 109 | | | domain |
| 17.006-13 | | | | | |
| 13.013-11 | | | | | |
| 15.025-01 | PWsp1429 | ATTCAACTCCATGCTCTTCT | CGG | Cas9CBE | Q > STOP L > F |
| 15.039-04 | | SEQ ID NO: 116 | | | |
| 17.006-13 | | | | | |
| 13.013-11 | | | | | |
| 15.025-01 | PWsp1403 | CGCTCTCCCACTGGGCCATG | TGG | Cas9CBE | W > STOP E > Y |
| 15.039-04 | | SEQ ID NO: 122 | | | |
| 17.006-13 | | | | | |
| 13.013-11 | | | | | |
| 15.025-01 | PWsp593 | TACATCCAAGAGTATGGCCA | TGG | Cas9CBE | Q > STOP, |
| 15.039-04 | | SEQ ID NO: 111 | | | Q > E |
| 17.006-13 | | | | | |

Cutting vectors targeting the MIXTA gene were constructed by inserted gRNAs into a CRISPR array. The gRNAs were designed to introduce a frameshift into the MIXTA genes and also target a known conserved region of the MIXTA gene family included the MYB domain necessary for function (see, e.g., see the MIXTA gene regions of SEQ ID NOs:77, 78, and 83-89).

TABLE 2
Blackberry lines, spacer sequences and edits

| | | | | | Change |
|---|---|---|---|---|---|
| Line | Spacer | Sequence | PAM | Enzyme | observed |
| 15.025-01 | PWsp657 | CCCAAAAGGACAGACAACGAGAT | TTTG | LbCas12a | indel |
| 15.039-04 | | SEQ ID NO: 53 | | | |
| 17.006-13 | | | | | |
| 13.013-11 | | | | | |
| 15.025-01 | PWsp658 | GGCAAATGATTTGCTATCGCTGA | TTTG | LbCas12a | indel |
| 15.039-04 | | SEQ ID NO: 54 | | | |
| 17.006-13 | | | | | |
| 13.013-11 | | | | | |
| 15.025-01 | PWsp659 | GTAGTTCTTGATCTCGTTGTCTG | TCCA | LbCas12a | indel |
| 15.039-04 | | SEQ ID NO: 55 | | | |
| 17.006-13 | | | | | |
| 13.013-11 | | | | | |
| 15.025-01 | PWsp660 | AGTAGTTCTTGATCTCGTTGTCT | TTCC | LbCas12a | indel |
| 15.039-04 | | SEQ ID NO: 56 | | | |
| 17.006-13 | | | | | |
| 15.025-01 | PWsp1163 | TCTTACATCCAAGAGTATGGCCA | TTTG | LbCas12a | indel |
| 15.039-04 | | SEQ ID NO: 110 | | | |
| 17.006-13 | | | | | |
| 13.013-11 | | | | | |
| 15.025-01 | PWsp1457 | ATATCAGGCCTCAGGTAATT | TTTG | LbCas12a | indel |
| 15.039-04 | | SEQ ID NO: 112 | | | |
| 17.006-13 | | | | | |
| 13.013-11 | | | | | |
| 15.025-01 | PWsp1458 | CTCTTTTGATATCAGGCCTC | TTTC | LbCas12a | indel |
| 15.039-04 | | SEQ ID NO: 113 | | | |
| 17.006-13 | | | | | |
| 13.013-11 | | | | | |
| 15.025-01 | PWsp1459 | GTTTGCAAGAAGAGCAGACT | TTTA | LbCas12a | indel |
| 15.039-04 | | SEQ ID NO: 114 | | | |
| 17.006-13 | | | | | |
| 13.013-11 | | | | | |
| 15.025-01 | PWsp1460 | CAAGAAGAGCAGACTATCAT | TTTG | LbCas12a | indel |
| 15.039-04 | | SEQ ID NO: 115 | | | |
| 17.006-13 | | | | | |
| 13.013-11 | | | | | |
| 15.025-01 | PWsp1461 | CGAGAAGAGCATGGAGTTGA | TTTC | LbCas12a | indel |
| 15.039-04 | | SEQ ID NO: 117 | | | |
| 17.006-13 | | | | | |
| 13.013-11 | | | | | |

TABLE 3
Strawberry line, spacer sequences and edits

| | | | | | Change |
|---|---|---|---|---|---|
| Line | Spacer | Sequence | PAM | Enzyme | observed |
| Hawaii 4 | PWsp1464 | CAGGAAGAGCAAACCATCAT | TTTG | LbCas12a | indel |
| | | SEQ ID NO: 123 | | | |
| Hawaii 4 | PWsp1458 | CTCTTTTGATATCAGGCCTC | TTTC | LbCas12a | indel |
| | | SEQ ID NO: 113 | | | |
| Hawaii 4 | PWsp338 | GCCGGCAAAGCACGCCAGCTTCC | TTTA | LbCas12a | indel |
| | | SEQ ID NO: 124 | | | |
| Hawaii 4 | PWsp1463 | GTTTGCAGGAAGAGCAAACC | TTTA | LbCas12a | indel |
| | | SEQ ID NO:125 | | | |
| Hawaii 4 | PWsp1465 | CCAGAAGAGCATGGAGTTGA | TTTC | LbCas12a | indel |
| | | SEQ ID NO: 126 | | | |
| Hawaii 4 | PWsp337 | ATATCAGGCCTCAGGTAGTTAGT | TTTG | LbCas12a | indel |
| | | SEQ ID NO: 127 | | | |
| Hawaii 4 | PWsp336 | AAACCCACCTTCTCGCAACATGG | TTTC | LbCas12a | indel |
| | | SEQ ID NO: 128 | | | |
| Hawaii 4 | PWsp1462 | CTCTTCCTGCAAACTAAACT | TTTG | LbCas12a | indel |
| | | SEQ ID NO: 129 | | | |
| Hawaii 4 | PWsp1466 | AAAAAAGGACCATGGACGCC | TTTG | LbCas12a | indel |
| | | SEQ ID NO: 130 | | | |

A vector encoding the spacer as described in the Tables 1-3, above, as well as a mutated Cas9 effector fused to a cytosine deaminase were introduced into explants.

### Example 5. Editing of MIXTA Genes in Plants

Different approaches were used to achieve edited MIXTA genes in the cells of blackberry, black raspberry and strawberry plants. In one example, the explants used were derived from the ‘regenerable callus’ system. In a further example, the explants were derived from the ‘nodal meristem’ system.

In each of these examples, the explants with successful DNA transfer were selected using an antibiotic or a fluorescent marker gene. The explants were grown on regeneration media that encourages shoot formation. After a healthy shoot forms, explants were transferred to rooting media that encourages root formation. Once both root and shoots have formed the plants are transferred initially to small pots in a growth chamber and then in bigger pots.

In some examples, explants were derived from a ‘hairy root’ system. For the hairy root system, cane nodes were first harvested from source plants, then sterilized and initiated into tissue culture. Infection of nodal explants was carried out with an Agrobacterium rhizogenes cell suspension. The explants are then transferred and cultured on induction medium without phytohormone for 4-6 weeks. Transgenic roots are identified by detecting reporter gene expression (e.g., expression of green fluorescent protein (GFP) gene). Composite plantlets containing transgenic roots and new axillary shoots are selected and then cultured in a plant tissue culture dish (e.g., Plantcon™). The composite plantlets are initially transplanted and grown in small pots in a growth chamber and then transplanted and grown in larger pots in a growth chamber. The mature roots are harvested and stored at about 5° C. for about 4 weeks for root induction. Transgenic plants are recovered from the roots through shoot initiation.

### Example 6. Editing Strawberry MIXTA

In one example, the explants used were derived from the ‘Strawberry petiole and leaves’ system. Explants for the strawberry transformation system were first harvested from source plants, then sterilized, injured, and initiated into tissue culture. Explants were then used for AB62 agrobacterium inoculation. Over the course of 3-4 weeks, through application of hormones, regenerable callus was induced, allowed to proliferate, and develop. Once a shoot emerges from the transgenic callus, a period for is allowed for shoot elongation (6-8 weeks).

The explants with DNA successful transfer were selected using hygromycin antibiotic and the presence of the fluorescent visual marker ZsGreen. The explants were grown on regeneration media that encourages shoot formation. After a healthy shoot has formed the explants are transferred to rooting media that encourages root formation. Once both root and shoots have formed, samples are taken for molecular analysis. the plants are transferred initially to small pots and maintained in a growth chamber and then bigger pots, destined for the greenhouse once next generation sequencing (NGS) data is available.

### Example 6. Molecular Screening

A PCR assay is used to molecularly confirm the putative transgenic plants. Then, the transgenic plants were assayed for edits at the MIXTA locus with standard NGS methods. The homozygous mutated plants were put forward for trait testing. The heterozygous plants were selfed to obtain the homozygous mutant in the subsequent generation and those progeny were then subjected to trait testing. The results showed over 150 edits using spacers PWsp593 and PWsp658 in the regions described herein, a selection of which are provided in Table 4. Additional spacers in these regions also provide endogenous MIXTA genes with edits.

TABLE 4
Analysis of edited MIXTA nucleic acid sequences (SEQ ID NOs: 101-108) obtain
in blackberry hairy root culture

| | | | Edit Position | |
|---|---|---|---|---|
| | | | Relative to | |
| | | | SEQ ID NO: | |
| Plant | | | 65 (BKB | |
| Identifier | Spacer | | MIXTA_Locus | |
| (SEQ ID NO) | Identifier | Edit Type | 3) | Edit |
| EPS4812 | PWsp593 | Replace- | 87-88 | CC > TG |
| (101) | | ment | | |
| EPS4366 | PWsp593 | Replace- | 87-88 | CC > TG |
| (101) | | ment | | |
| EPS4313 | PWsp593 | Replace- | 87-88 | CC > TT |
| (102) | | ment | | |
| EPS4309A | PWsp593 | Deletion | After 86 | 7 bp deletion |
| (103) | | | | CCAAGAG |
| EPS4309B | PWsp593 | Deletion | After 88 | 11 bp deletion |
| (104) | | | | AAGAGTATGGG |
| | | | | (SEQ ID NO: 118) |
| EPS4309C | PWsp593 | Deletion | After 86 | 13 bp deletion |
| (105) | | | | CCAAGAGTATGGG |
| | | | | (SEQ ID NO: 119) |
| EPS4309D | PWsp593 | Deletion | After 77 | 23 bp deletion |
| (106) | | | | GTCTTACATCCAAGAGTATGGGC |
| | | | | (SEQ ID NO: 120) |
| EPS4472 | PWsp593 | Deletion | After 268 | 7 bp deletion |
| (107) | | | | CAGCGAT |
| EPS4281 | PWsp658 | Deletion | After 269 | 7 bp deletion |
| (108) | | | | AGCGATA |

### Example. 7. Trait Testing

Morphological studies of developing Rubus prickles suggest that they are modified glandular trichomes. Additionally, the correlation between the presence of glandular trichomes and the presence of prickles in blackberry, for example, suggest that glandular trichomes are indeed involved in blackberry prickle development. We, therefore, used the presence of glandular trichomes in tissue culture to first score the plants.

For this purpose, an expanded leaf is removed from a plant by cutting at the base of the petiole. Using a scalpel and tweezers, the leaf is removed. The leaf is then observed under a dissecting microscope to look for glandular trichomes. Blackberry glandular trichomes are typically about 1 mm in length and have a defined glandular head. In some cases, they can be partially obscured by hairy trichomes due to their short length. After examining the glandular trichome, presence is assessed as: 1=hairy trichomes only, 4=short glandular trichomes only, 7=short glandular trichomes are present elongated glandular trichomes are present but not abundant, 9=short and elongated glandular trichomes are abundant.

After the plants have grown, the canes are examined. The presence of any prickles or hairlike bristles on the cane are noted. The prickle type is scored in the following manner: 1=no bristles or prickles, 3=short hairlike bristles (1-2 mm in length) but no prickles, 5=elongated hairlike bristles (>2 mm in length) but no prickles, 7=prickles but no hairlike bristles. 9=both hairlike bristles and prickles.

The foregoing is illustrative of the present invention and is not to be construed as limiting thereof. The invention is defined by the following claims, with equivalents of the claims to be included therein.

That which is claimed is:

1. A Rubus plant or plant part thereof comprising at least one non-natural mutation in at least one copy of an endogenous gene encoding a MIXTA transcription factor.

2. The Rubus plant or part thereof of claim 1, wherein the at least one non-natural mutation is a null allele or is a dominant negative mutation.

3. The Rubus plant or part thereof of claim 1 or claim 2, wherein the MIXTA transcription factor gene is capable of regulating thorn or prickle production.

4. The Rubus plant or part thereof of any one of claims 1-3, wherein the MIXTA transcription factor comprises the amino acid sequence of SEQ ID NOs: 57-62, 71-76, 90-92, 94, 96, 98, or 100 or a sequence having at least at least 94% identity to any one of the amino acid sequences of SEQ ID NO:57.

5. The Rubus plant or part thereof of any of the preceding claims, wherein the endogenous gene encoding a MIXTA transcription factor comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83.

6. The Rubus plant or part thereof of any of the preceding claims, wherein the plant or plant part comprises at least two (at least four, at least six, at least eight, at least ten, at least twelve) copies of the endogenous gene encoding a MIXTA transcription factor and each copy comprises at least one non-natural mutation that is a null allele or a dominant negative mutation.

7. The Rubus plant or part thereof of any of the preceding claims, wherein the at least one non-natural mutation is a base substitution, a deletion and/or an insertion.

8. The Rubus plant or part thereof of any of the preceding claims, wherein the at least one non-natural mutation comprises a base substitution to an A, a T, a G, or a C.

9. The Rubus plant or part thereof of any of the preceding claims, wherein the at least one non-natural mutation is a deletion

10. The Rubus plant or part thereof of claim 9, wherein the deletion is a truncation.

11. The Rubus plant or part thereof of claim 9 or claim 10, wherein the deletion is C-terminal truncation.

12. The Rubus plant or part thereof of claim 11, wherein the C-terminal truncation comprises a truncation of at least 1 amino acid residue to about 350 consecutive amino acid residues from the C-terminus of the MIXTA transcription factor.

13. The Rubus plant or part thereof of claim 9, wherein the deletion comprises a deletion of at least 5 consecutive base pairs to about 1200, 1210, 1220, 1230, 1240 or 1250 consecutive base pairs from the endogenous gene encoding a MIXTA transcription factor.

14. The Rubus plant or part thereof of any one of the preceding claims, wherein the plant comprising the at least one non-natural mutation is thornless/prickleless or substantially thornless/prickleless.

15. The Rubus plant or part thereof of any one of the preceding claims, wherein the Rubus plant is a blackberry, black raspberry or raspberry.

16. The Rubus plant or part thereof of any one of the preceding claims, wherein the Rubus plant is a blackberry plant and the endogenous MIXTA transcription factor gene is present in two copies, four copies, six copies or eight copies, and each copy comprises the at least one non-natural mutation in the endogenous gene encoding a MIXTA transcription factor.

17. The Rubus plant or part thereof of any one of the preceding claims, wherein the Rubus plant or part thereof further comprises a non-natural mutation in an endogenous AG Glade MADS-box transcription factor gene, optionally in an endogenous AG clade MADS-box transcription factor gene encoding AGAMOUS (AG), SHATTERPROOF 1 (SHP1), SHATTERPROOF 2 (SHP2), and/or SEEDSTICK (STK).

18. The Rubus plant or part thereof of any one of the preceding claims, wherein the Rubus plant or part thereof further comprises a non-natural mutation in an endogenous gene encoding SEEDSTICK (STK).

19. The Rubus plant or part thereof of claim 17 or claim 18, wherein the plant is thornless/prickleless or substantially thornless/prickleless (has reduced thorns and/or reduced prickleness) and seedless or has reduced seediness

20. A Rubus plant cell, comprising an editing system comprising:
(a) a CRISPR-associated effector protein; and
(b) a guide nucleic acid having a spacer sequence with complementarity to an endogenous target gene encoding a wild type MIXTA transcription factor.

21. The Rubus plant cell of claim 20, wherein the wild type MIXTA transcription factor comprises the amino acid sequence of SEQ ID NOs:57-62, 71-76, 90-92, 94, 96, 98, or 100 or an amino acid sequence having at least at least 94% identity to SEQ ID NO:57.

22. The Rubus plant cell of claim 21, wherein the guide nucleic acid comprises a nucleotide sequence of any one of SEQ ID NOs:53-56 or 109-117.

23. A Rubus plant cell comprising at least one non-natural mutation within an endogenous MIXTA transcription factor gene, wherein the mutation is a substitution, insertion or a deletion that is introduced using an editing system that comprises a nucleic acid binding domain that binds to a target site in the endogenous MIXTA transcription factor gene, wherein the endogenous MIXTA transcription factor gene comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83 or encodes a polypeptide comprising the sequence of any one of the amino acid sequences of SEQ ID NOs:57-62, 71-76, 90-92, 94, 96, 98, or 100 or a sequence having at least at least 94% identity to any one of the amino acid sequences of SEQ ID NO:57.

24. The Rubus plant cell of claim 23, wherein the mutation is a null allele or a dominant negative mutation.

25. The Rubus plant cell of claim 23 or claim 24 wherein the mutation is a deletion.

26. The Rubus plant cell of claims 23-25, wherein the deletion is a C-terminal truncation comprising a truncation of at least 1 amino acid residue to about 400 consecutive amino acid residues from the C-terminus of the polypeptide comprising a sequence of any one of the amino acid sequences of SEQ ID NOs:57-62, 71, 72, 94, 96, 98, or 100 or a sequence having at least at least 94% identity to any one of the amino acid sequences of SEQ ID NO:57.

27. The Rubus plant cell of any one of claims 23-26, wherein the mutation is a deletion of at least 5 consecutive base pairs to about 1200, 1210, 1220, 1230, 1240 or 1250 consecutive base pairs from a nucleotide sequence of any one of SEQ ID NOs:63-70, 93, 95, 97, or 99, a sequence comprising the nucleotide sequence of SEQ ID NOs:77 or 84-89 or a sequence having at least 95% identity to the nucleotide sequence of SEQ ID NO:78 or SEQ ID NO:83.

28. The Rubus plant cell of any one of claims 23-27, wherein the mutation is made following cleavage by the editing system, wherein the editing system further comprises a nuclease and the DNA-binding domain binds to a target site binds to a target site within a target nucleic acid comprising the nucleotide sequence of SEQ ID NO:77 or 84-89, or a sequence having at least 95% identity to the nucleotide sequence of SEQ ID NO:78 or SEQ ID NO:83 or encoding a polypeptide comprising the sequence of any one of the amino acid sequences of SEQ ID NOs:73-76 or 90-92.

29. The Rubus plant cell of claim 28, wherein the nuclease is a zinc finger nuclease, transcription activator-like effector nucleases (TALEN), endonuclease (e.g., Fok1) or a CRISPR-Cas effector protein.

30. The Rubus plant cell of claim 28 or claim 29, wherein the DNA binding domain is a zinc finger, transcription activator-like DNA binding domain (TAL), argonaute or a CRISPR-Cas effector DNA binding domain.

31. The Rubus plant cell of any one of claims 20-30, wherein the Rubus plant cell is from a blackberry plant, black raspberry plant or raspberry plant.

32. The Rubus plant cell of any one of claims 23-30, wherein the Rubus plant cell is from a blackberry plant and the cell comprises two copies, four copies, six copies or eight copies of the endogenous MIXTA transcription factor gene, and each copy is edited and comprises the at least one non-natural mutation.

33. The Rubus plant cell of any one of claims 23-32, wherein the Rubus plant or part thereof further comprises a non-natural mutation in an endogenous AG clade MADS-box transcription factor gene, optionally in an endogenous AG clade MADS-box transcription factor gene encoding AGAMOUS (AG), SHATTERPROOF 1 (SHP1), SHATTERPROOF 2 (SHP2), and/or SEEDSTICK (STK).

34. The Rubus plant cell of any one of claims 23-32, wherein the Rubus plant cell further comprises a non-natural mutation in an endogenous gene encoding SEEDSTICK (STK).

35. A Rubus plant regenerated from the plant part of any one of claims 1 to 16, or the plant cell of claims 20 to 34.

36. The Rubus plant of claim 35, wherein the plant comprising the mutation is thornless/prickleless or substantially thornless/prickleless (has reduced thorns and/or reduced prickleness).

37. A Rubus plant regenerated from the plant part of any one of claim 17 or 18, or the plant cell of any one of any one of claims 33 to 34.

38. The Rubus plant of claim 37, wherein the plant comprising the mutation is thornless/prickleless or substantially thornless/prickleless (has reduced thorns and/or reduced prickleness) and seedless or has reduced seediness.

39. A method of producing/breeding a transgene-free edited Rubus plant, comprising:
crossing the Rubus plant of any one of the preceding claims with a transgene free Rubus plant, thereby introducing the at least one non-natural mutation into the Rubus plant that is transgene-free; and
selecting a progeny Rubus plant that comprises the at least one non-natural mutation and is transgene-free, thereby producing a transgene free edited Rubus plant.

40. A method of providing a plurality of Rubus plants having reduced thorns and/or reduced prickleness, the method comprising planting two or more plants of any one of claim 1-19 or 35-38 in a growing area (e.g., an agricultural field, greenhouse, and the like), thereby providing a plurality of Rubus plants having reduced thorns and/or reduced prickleness as compared to a plurality of control Rubus plants not comprising the mutation.

41. A method for editing a specific site in the genome of a Rubus plant cell, the method comprising cleaving, in a site specific manner, a target site within an endogenous MIXTA transcription factor gene in the Rubus plant cell, the endogenous MIXTA transcription factor gene comprising the nucleotide sequence of SEQ ID NOs:63-70, 77, 84-89, 93, 95, 97, or 99 or a sequence having at least 95% identity to the nucleotide sequence of SEQ ID NO:78 or SEQ ID NO:83 or encoding a polypeptide comprising the sequence of any one of the amino acid sequences of SEQ ID NOs: NOs:57-62, 71-76, 90-92, 94, 96, 98, or 100 or a sequence having at least at least 94% identity to any one of the amino acid sequences of SEQ ID NO:57, thereby generating an edit in the endogenous MIXTA transcription factor gene of the Rubus plant cell and producing a plant cell comprising the edit in the endogenous MIXTA transcription factor gene.

42. The method of claim 41, wherein the Rubus plant cell is from a blackberry plant, black raspberry plant or raspberry plant.

43. The method of claim 41, wherein the Rubus plant cell is from a blackberry plant and the endogenous MIXTA transcription factor gene is present in the cell in two copies, four copies, six copies or eight copies, each copy comprising the edit in the endogenous MIXTA transcription factor gene.

44. The method of any one of claims 41-43, further comprising regenerating a Rubus plant from the Rubus plant cell comprising the edit in the endogenous MIXTA transcription factor gene, thereby producing a Rubus plant comprising the edit in the endogenous MIXTA transcription factor gene.

45. The method of claim 44, wherein the Rubus plant comprising the edit in the endogenous MIXTA transcription factor gene has reduced thorns and/or prickles compared to a control Rubus plant that does not comprise the edit.

46. The method of any one of claims 41-45, wherein the edit results in a non-natural mutation.

47. The method of claim 46, wherein the mutation is a null allele or a dominant negative mutation.

48. The method of claim 46 or claim 47, wherein the mutation is a deletion.

49. The method of claim 48, wherein the deletion is a C-terminal truncation comprising a truncation of at least 1 amino acid residue to about 400 consecutive amino acid residues from the C-terminus of the polypeptide comprising a sequence of any one of the amino acid sequences of SEQ ID NOs:57-62, 71, 72, 94, 96, 98, or 100 or a sequence having at least at least 94% identity to any one of the amino acid sequences of SEQ ID NO:57.

50. The method of any one of claims 46-48, wherein the mutation is a deletion of at least 5 consecutive base pairs to about 1200, 1210, 1220, 1230, 1240 or 1250 consecutive base pairs from a nucleotide sequence of any one of SEQ ID NOs:63-70, 93, 95, 97, or 99, a sequence comprising the nucleotide sequence of SEQ ID NO:77 or 84-89, or a sequence having at least 95% identity to the nucleotide sequence of SEQ ID NO:78 or SEQ ID NO:83.

51. A method for making a Rubus plant, comprising:
(a) contacting a population of Rubus plant cells comprising at least one wild type endogenous MIXTA transcription factor gene with a nuclease linked to a DNA binding domain (e.g., an editing system) that binds to a target site in the at least one wild type endogenous MIXTA transcription factor gene,
wherein the at least one wild type endogenous MIXTA transcription factor gene:
(i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or
(ii) comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83;
(b) selecting a Rubus plant cell from said population that comprises a mutation in the at least one wild type endogenous MIXTA transcription factor gene; and
(c) growing the selected Rubus plant cell into a Rubus plant comprising the mutation in at least one endogenous MIXTA transcription factor gene.

52. A method for reducing thorns and prickles in a Rubus plant or part thereof, comprising
(a) contacting a Rubus plant cell comprising a wild type endogenous MIXTA transcription factor gene with a nuclease targeting the wild type endogenous MIXTA transcription factor gene, wherein the nuclease is linked to a DNA binding domain that binds to a target site in the wild type endogenous MIXTA transcription factor gene,
wherein the wild type endogenous MIXTA transcription factor gene:
(i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or
(ii) comprises a nucleotide sequence of any one of SEQ ID NOs:63-70, 77, 84-89, 93, 95, 97, or 99 or a sequence having at least 95% identity to the nucleotide sequence of SEQ ID NO:78 or SEQ ID NO:83;
(b) growing the plant cell into a plant, thereby reducing thorns and prickles in the Rubus plant, thereby reducing thorns and prickles in the Rubus plant or part thereof.

53. The method of claim 51 or claim 52, wherein the population of Rubus plant cells or the Rubus plant cell is/are from a blackberry plant, black raspberry plant or raspberry plant.

54. The method of claim 51 or claim 52, wherein the population of Rubus plant cells or the Rubus plant cell is/are from a blackberry plant and the endogenous MIXTA transcription factor gene is present in each cell in two copies, four copies, six copies or eight copies, each of which is edited to have at least one non-natural mutation.

55. A method for producing a Rubus plant or part thereof comprising at least one cell having a mutated endogenous MIXTA transcription factor gene, the method comprising
contacting a target site in an endogenous MIXTA transcription factor gene in the Rubus plant or plant part with a nuclease comprising a cleavage domain and a DNA-binding domain, wherein the DNA binding domain binds to a target site in the endogenous MIXTA transcription factor gene,
wherein the endogenous MIXTA transcription factor gene (i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or (ii) comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83, thereby producing the Rubus plant or part thereof comprising at least one cell having a mutation in the endogenous MIXTA transcription factor gene.

56. A method for producing a Rubus plant or part thereof comprising a mutated endogenous MIXTA transcription factor and reduced thorns and/or prickles, the method comprising
contacting a target site in an endogenous MIXTA transcription factor gene in the Rubus plant or plant part with a nuclease comprising a cleavage domain and a DNA-binding domain, wherein the DNA binding domain binds to a target site in the endogenous MIXTA transcription factor gene,
wherein the endogenous MIXTA transcription factor gene:
(i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or
(ii) comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83, thereby producing the Rubus plant or part thereof comprising a mutated endogenous MIXTA transcription factor and reduced thorns and/or prickles.

57. The method of any one of claims 52-56, wherein the nuclease cleaves the endogenous MIXTA transcription factor gene and a mutation is introduced into the endogenous MIXTA transcription factor gene.

58. The method of claim 57, wherein the mutation is a non-naturally occurring mutation.

59. The method of claim 58, wherein the mutation results in a null allele or a dominant negative mutation in the endogenous MIXTA transcription factor gene

60. The method of any one of claims 57-59, wherein the mutation is a substitution, an insertion and/or a deletion.

61. The method of any one of claims 57-60, wherein the mutation is a deletion.

62. The method of claim 60 or claim 61, wherein the deletion is a truncation.

63. The method of claim 62, wherein the truncation is a C-terminal truncation comprising a truncation of a polypeptide comprising any one of the amino acid sequences of SEQ ID NOs:57-62, 71, 72, 94, 96, 98, or 100 or a sequence having at least at least 94% identity to any one of the amino acid sequences of SEQ ID NO:57.

64. The method of claim 63, wherein the C-terminal truncation comprises a deletion of at least 1 amino acid residue to about 400 consecutive amino acid residues.

65. The method of claim 60 or claim 61, wherein the mutation is a deletion of at least 5 consecutive base pairs to about 1200, 1210, 1220, 1230, 1240 or 1250 consecutive base pairs from a nucleotide sequence of any one of SEQ ID NOs:63-70, 93, 95, 97, or 99, a sequence comprising the nucleotide sequence of SEQ ID NO:77 or 84-89, or a sequence having at least 95% identity to the nucleotide sequence of SEQ ID NO:78 or SEQ ID NO:83.

66. The method of any one of claims 55-65, wherein the Rubus plant is a blackberry, black raspberry or raspberry.

67. The method of any one of claims 55-65, wherein the Rubus plant is a blackberry plant and the blackberry plant comprises the endogenous MIXTA transcription factor gene in two copies, four copies, six copies or eight copies, and each copy of the endogenous MIXTA transcription factor gene is edited and comprises a non-natural mutation.

68. The method of any one of claims 55-67, wherein the Rubus plant or part thereof further comprises a non-natural mutation in an endogenous AG clade MADS-box transcription factor gene, optionally in an endogenous AG clade MADS-box transcription factor gene encoding AGAMOUS (AG), SHATTERPROOF 1 (SHP1), SHATTERPROOF 2 (SHP2), and/or SEEDSTICK (STK).

69. The method of any one of claims 55-67, wherein the Rubus plant or part thereof further comprises a non-natural mutation in an endogenous gene encoding SEEDSTICK (STK).

70. The method of claim 68 of claim 69, wherein the Rubus plant is thornless/prickleless or substantially thornless/prickleless (has reduced thorns and/or reduced prickleness) and seedless or has reduced seediness.

71. The method of any one of claims 52-70, wherein the nuclease is a zinc finger nuclease, transcription activator-like effector nucleases (TALEN), endonuclease (e.g. Fok1) or a CRISPR-Cas effector protein.

72. The method of any one of claims 52-71 wherein the DNA binding domain is a zinc finger, transcription activator-like DNA binding domain (TAL), argonaute or a CRISPR-Cas effector DNA binding domain.

73. A guide nucleic acid (e.g., gRNA, gDNA, crRNA, crDNA) that binds to a target site in a MIXTA transcription factor gene, wherein the endogenous MIXTA transcription factor gene:
(i) encodes a polypeptide comprising the sequence of any one of the amino acid sequences of SEQ ID NOs: 57-62, 71-76, 90-92, 94, 96, 98, or 100 or a sequence having at least at least 94% identity to any one of the amino acid sequences of SEQ ID NO:57; and/or
(ii) comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83.

74. The guide nucleic acid of claim 73, wherein the guide nucleic acid comprises a spacer having the nucleotide sequence of any one of SEQ ID NOs: 53-56 or 109-117.

75. A system comprising the guide nucleic acid of claim 73 or claim 74 and a CRISPR-Cas effector protein that associates with the guide nucleic acid.

76. The system of claim 75, further comprising a tracr nucleic acid that associates with the guide nucleic acid and a CRISPR-Cas effector protein, optionally wherein the tracr nucleic acid and the guide nucleic acid are covalently linked.

77. A gene editing system comprising a CRISPR-Cas effector protein in association with a guide nucleic acid, wherein the guide nucleic acid comprises a spacer sequence that binds to a MIXTA transcription factor gene.

78. The gene editing system of claim 77, wherein the MIXTA transcription factor gene comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83 or encodes a polypeptide comprising the sequence of any one of the amino acid sequences of SEQ ID NOs: 57-62, 71-76, 94, 96, 98, or 100 or a sequence having at least at least 94% identity to any one of the amino acid sequences of SEQ ID NO:57.

79. The gene editing system of claim 77 or claim 78, wherein the guide nucleic acid comprises a spacer sequence having the nucleotide sequence of any one of SEQ ID NOs:53-56 or 109-117.

80. The gene editing system of any one of claims 77-79, further comprising a tracr nucleic acid that associates with the guide nucleic acid and a CRISPR-Cas effector protein, optionally wherein the tracr nucleic acid and the guide nucleic acid are covalently linked.

81. A complex comprising a CRISPR-Cas effector protein comprising a cleavage domain and a guide nucleic acid, wherein the guide nucleic acid binds to a target site in a MIXTA transcription factor gene, wherein the MIXTA transcription factor gene comprises a nucleotide sequence of any one of SEQ ID NOs: 63-70, 77, 84-89, 93, 95, 97, or 99 or a sequence having at least 95% identity to the nucleotide sequence of SEQ ID NO:78 or SEQ ID NO:83 or encodes a polypeptide comprising the sequence of any one of the amino acid sequences of SEQ ID NOs: 57-62, 71-76, 94, 96, 98, or 100 or a sequence having at least at least 94% identity to any one of the amino acid sequences of SEQ ID NO:57, wherein the cleavage domain cleaves a target strand in the MIXTA transcription factor gene.

82. An expression cassette comprising (a) a polynucleotide encoding CRISPR-Cas effector protein comprising a cleavage domain and (b) a guide nucleic acid that binds to a target site in an endogenous MIXTA transcription factor gene, wherein the guide nucleic acid comprises a spacer sequence that is complementary to and binds: (i) to a portion of the nucleotide sequence of SEQ ID NO:77 or SEQ ID NO:84-89 or a nucleotide sequence having at least 95% identity to the nucleotide sequence of SEQ ID NO:78 or SEQ ID NO:83 or (ii) to a portion of a nucleotide sequence encoding any one of the amino acid sequences of SEQ ID NOs:73-76 or 90-92.

83. A nucleic acid encoding a null allele or a dominant negative mutation of an endogenous MIXTA transcription factor gene.

84. A Rubus plant or part thereof comprising the nucleic acid of claim 83.

85. The Rubus plant or part thereof of claim 84, comprising reduced thorns and/or prickles.

86. The Rubus plant or part thereof of claim 84 or claim 85, wherein the Rubus plant is a blackberry, black raspberry, or raspberry.

87. The Rubus plant or part thereof of any one of claims 84-86, wherein the Rubus plant or part thereof further comprises a non-natural mutation in an endogenous AG clade MADS-box transcription factor gene, optionally in an endogenous AG clade MADS-box transcription factor gene encoding AGAMOUS (AG), SHATTERPROOF 1 (SHP1), SHATTERPROOF 2 (SHP2), and/or SEEDSTICK (STK).

88. The Rubus plant or part thereof of any one of claims 84-86, wherein the Rubus plant or part thereof further comprises a non-natural mutation in an endogenous gene encoding SEEDSTICK (STK).

89. The Rubus plant of claim 87 or claim 88, wherein the plant is thornless/prickleless or substantially thornless/prickleless (has reduced thorns and/or reduced prickleness) and is seedless or has reduced seediness.
